(19) United States
(12) Patent Application Publication
(10) Pub. No.: US 2021/0238632 A1
(43) Pub. Date: Aug. 5, 2021

(54) VECTOR PRODUCTION IN SERUM FREE MEDIA

(71) Applicant: CSL Behring Gene Therapy, Inc., Pasadena, CA (US)

(72) Inventors: Jeffrey S. Bartlett, Columbus, OH (US); Chi-Lin Lee, Arcadia, CA (US)

(21) Appl. No.: 17/177,239

(22) Filed: Feb. 17, 2021

Related U.S. Application Data
(63) Continuation of application No. PCT/US2019/047800, filed on Aug. 23, 2019.
(60) Provisional application No. 62/722,784, filed on Aug. 24, 2018.

(51) Int. Cl. C12N 15/86; C12N 7/00; C12N 15/113; A61K 38/55; A61K 38/45; A61K 38/17

(52) U.S. Cl. CPC C12N 15/86; C12N 7/00; C12N 15/1137; C12N 15/1138; A61K 38/55; C12Y 207/10002; A61K 38/1722; C12N 2500/99; C12N 2310/531; C12Y 204/02008; A61K 38/45

(57) ABSTRACT

In one aspect of the present disclosure is a method of harvesting viral titer about every 40 hours to about every 56 hours following induction of stable producer cell line cells, wherein the viral titer is at least partially harvested in a serum-free medium. In another aspect of the present disclosure is a method of harvesting vector supernatant comprising: generating stable producer cell line cells; inducing viral vector production from the generated stable producer cell line cells; and repeatedly harvesting the viral vectors from the induced generated stable producer cell line cells in serum-free media every about 40 to about 56 hours following an initial harvesting of the viral vectors.

### CROSS REFERENCE TO RELATED APPLICATIONS

The present application is a continuation of International Application No. PCT/US2019/047800 filed on Aug. 23, 2019, which application claims the benefit of the filing date of U.S. Provisional Patent Application No. 62/722,784 filed on Aug. 24, 2018, the disclosures of which are hereby incorporated by reference herein in their entireties.

### FIELD OF DISCLOSURE

The present disclosure generally relates to bio-manufacturing methods and methods of harvesting viral titer.

### BACKGROUND OF THE DISCLOSURE

Cell lines and primary cells grown under in vitro culturing conditions require a special growth and maintenance medium that can support (i) cell replication in the logarithmic phase, and (i) cell maintenance once the cells are no longer dividing, i.e., when the cells are in the stationary phase. Commonly used cell culture media comprise a rich salt solution containing vitamins, amino acids, essential trace elements and sugars. Growth hormones, enzymes and biologically active proteins required for supporting cell growth and maintenance are usually added as a supplement to the medium in the form of an animal blood derived serum product. Examples of animal blood derived serum products are fetal calf serum, chicken serum, horse serum and porcine serum. These sera are derived from fractionated blood, from which the red blood cells and the white blood cells have been removed.

The animal sera not only comprise factors that are required for the growth of cells, but also factors that are required for cells that naturally grow as adherent cells to attach to the cell support surface of the culture vessel. Thus, it is critical for adherent cells that enough serum is added to the medium to enable them to grow and form a monolayer.

Unfortunately, bovine/fetal calf serum, as well as sera from other animals, may contain adventitious pathogenic agents such as viruses or prion proteins. There is a potential risk that these pathogenic agents may be transmitted to the animal/human to be treated or vaccinated with the vaccine or any other pharmaceutical product produced in cell culture. This is of particular relevance if cell culture products are administered to immune-compromised humans. In view of the possible risk associated with the use of animal sera in cell culture it has become clear that manufacturing processes free from the use of animal products are highly desirable.

Producing self-inactivating-vectors (“SIN-vectors”) at scales to support clinical trials is an important challenge within the field. While gamma-retroviral vectors can be produced by either transient transfection or the generation of stable producer cell lines, lentiviruses require the expression of multiple cytotoxic accessory genes, which makes the generation of producer cells more complicated (Greene et al., Transduction of Human CD34+ Repopulating Cells with a Self-Inactivating Lentiviral Vector for SCID-Xl Produced at Clinical Scale by a Stable Cell Line, HGTM, 23, 297-308 (October 2012), the disclosure of which is hereby incorporated by reference in its entirety). Transient transfection is instead the current technology for pilot production of lentivirus, which is impractical for very large-scale applications under a safety, cost, and reproducibility standpoint. In fact, this technology is expensive, is difficult to standardize and scale-up, and suffers from batch-to-batch variability and low reverse transcriptase fidelity (Stornaiuolo et al., RD2-MolPack-Chim3, a Packaging Cell Line for Stable Production of Lentiviral Vectors for Anti-HIV Gene Therapy, HGTM, 24:228-240 (August 2013), the disclosure of which is hereby incorporated by reference in its entirety).

### BRIEF SUMMARY OF THE DISCLOSURE

While a production protocol utilizing a daily harvest (e.g. harvest every 24 hours) may be utilized to produce a variety of test vectors at small scale, daily harvesting and media exchange is often not economical. As an alternative to daily harvesting, Applicant has developed a “two-day harvest” protocol where viral vectors are repeatedly harvested in serum-free media every about 40 hours to about 56 hours following an initial harvesting of the viral vectors. Applicant has unexpectedly discovered that such a “two-day harvest” protocol using serum-free media allows for the generation of about the same quantity of viral vectors as with the more traditional daily harvest, while also providing the benefit of requiring less culture medium. It is believed that the use of a serum-free medium together with the disclosed “two-day harvest” procedure is especially important for large-scale bio-manufacturing given the high costs associated with serum-containing media as compared with serum-free media. Additionally, the risk of transmitting pathogenic agents, potentially present in serum-containing media, to subjects treated with the harvested viral vectors is minimized or eliminated when using serum-free media. These benefits satisfy the unmet need in the industry of reducing large-scale biomanufacturing costs and enhancing safety, while at the same time providing a bio-manufacturing process where high quantities of viral vector titer may be recovered.

In a first aspect of the present disclosure is a method of harvesting vector supernatant comprising: generating stable producer cell line cells; inducing viral vector production from the generated stable producer cell line cells; and repeatedly harvesting the viral vectors from the induced generated stable producer cell line cells in serum-free media every about 40 to about 56 hours following an initial harvesting of the viral vectors. In some embodiments, the repeated harvesting comprises adding fresh serum-free media to the induced generated stable producer cell line cells without introducing additional generated stable producer cell line cells.

In some embodiments, the serum-free media used for harvesting is replaced after each repeated harvesting. In some embodiments, no additional serum-free media is introduced to the generated stable producer cell line cells during each individual harvesting. In some embodiments, the serum-free media includes one or more growth factors. In some embodiments, the serum-free media includes one or more lipids. In some embodiments, the serum-free media includes both growth factors and lipids. In some embodiments, the lipids include cholesterol, phospholipids, and fatty acids.

In some embodiments, the initial harvesting of the viral vectors occurs between about 40 hours to about 56 hours after induction (i.e. after inducing viral vector production). In some embodiments, the initial harvesting of the viral vectors occurs less than 48 hours after induction. In some embodiments, the repeated harvesting of the viral vectors occurs every about 44 to about 52 hours. In some embodiments, the repeated harvesting of the viral vectors occurs every about 46 to about 50 hours. In some embodiments, the repeated harvesting of the viral vectors occurs every about 48 hours.

In some embodiments, the method provides for a production of viral titer ranging from between about 0.5×10⁶ TU/mL to about 4×10⁶ TU/mL during each individual harvesting of the repeated harvesting. In some embodiments, the viral titer ranges from between about 0.5×10⁶ TU/mL to about 2×10⁶ TU/mL during each individual harvesting of the repeated harvesting. In some embodiments, the viral titer ranges from between about 0.5×10⁶ TU/mL to about 1.5×10⁶ TU/mL during each individual harvesting of the repeated harvesting. In some embodiments, the repeated harvesting of the viral vectors occurs at least twice. In some embodiments, the repeated harvesting of the viral vectors occurs at least three times. In some embodiments, the repeated harvesting of the viral vectors occurs at least four times. In some embodiments, the repeated harvesting of the viral vectors occurs at least 5 times. In some embodiments, the repeated harvesting of the viral vectors occurs at least 10 times. In some embodiments, the repeated harvesting of the viral vectors occurs at least 20 times. In some embodiments, the repeated harvesting occurs for a period of time ranging from between about 10 days to about 90 days. In some embodiments, the repeated harvesting occurs for a period of time ranging from between about 20 days to about 70 days.

In some embodiments, the stable producer cell line cells are derived from packaging cell line cells. In some embodiments, the packaging cell line cells are derived from cells selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In some embodiments, the packaging cell line cells are selected from the group consisting of GPR, GPRG, GPRT, GPRGT, and GPRT-G cell line cells.

In some embodiments, the stable producer cell line cells are generated by (a) synthesizing a vector by cloning one or more genes into a recombinant plasmid; (b) forming a concatemeric array from (i) an expression cassette excised from the synthesized vector, and (ii) an expression cassette obtained from an antibiotic resistance cassette plasmid; (c) transfecting GPR, GPRG, GPRT, GPRGT, or GPRT-G packaging cell line cells with the formed concatemeric array; and (d) isolating the stable producer cell line cells. In some embodiments the synthesized vector is a lentiviral vector, e.g. a self-inactivating lentiviral vector. In some embodiments, the antibiotic resistance cassette plasmid is a bleomycin antibiotic resistance cassette. In some embodiments, a molar ratio of the expression cassette excised from the synthesized vector and the expression cassette obtained from the bleomycin antibiotic resistance cassette ranges from between about 50:1 to about 1:50. In some embodiments, the molar ratio ranges from between about 25:1 to about 1:25. In some embodiments, the molar ratio ranges from between about 15:1 to about 1:15. In some embodiments, the molar ratio ranges from about 10:1 to about 1:10.

In some embodiments, the recombinant plasmid comprises a nucleotide sequence having at least about 90% identity to that of SEQ ID NO: 1. In some embodiments, the recombinant plasmid comprises a nucleotide sequence having at least about 95% identity to that of SEQ ID NO: 1. In some embodiments, the recombinant plasmid comprises a nucleotide sequence having at least about 99% identity to that of SEQ ID NO: 1. In some embodiments, the recombinant plasmid comprises a nucleotide sequence having at least about 90% identity to that of SEQ ID NO: 2. In some embodiments, the recombinant plasmid comprises a nucleotide sequence having at least about 95% identity to that of SEQ ID NO: 2. In some embodiments, the recombinant plasmid comprises a nucleotide sequence having at least about 99% identity to that of SEQ ID NO: 2. In some embodiments, the recombinant plasmid comprises a multiple cloning site having BstBI, MluI, NotI, and/or ClaI restriction endonuclease sites. In some embodiments, a nucleotide sequence encoding the multiple cloning site has at least about 90% sequence identity to that of SEQ ID NO: 7. In some embodiments, a nucleotide sequence encoding the multiple cloning site has at least about 95% sequence identity to that of SEQ ID NO: 7. In some embodiments, the recombinant plasmid further comprises a nucleotide sequence encoding a packaging signal; a nucleotide sequence encoding a central polypurine tract; a nucleotide sequence encoding a Rev response element; and a nucleotide sequence encoding a self-inactivating long terminal repeat. In some embodiments, the vector cassette is flanked by at least two additional restriction endonuclease sites, the at least two additional restriction endonuclease sites independently selected from sfiI and Bsu36I.

In some embodiments, the synthesized vector includes a nucleic acid sequence encoding a therapeutic gene (including any of those genes enumerated herein). In some embodiments, the therapeutic gene corrects for sickle cell disease or at least mitigates one symptom of a sickle cell disease. In some embodiments, the therapeutic gene is a gamma-globin gene. In some embodiments, the therapeutic gene is a Wiskott-Aldrich Syndrome protein. In some embodiments, the therapeutic gene is a C1 esterase inhibitor protein. In some embodiments, the therapeutic gene is Bruton's tyrosine kinase. In some embodiments, the synthesized vector comprises a nucleic acid sequence to knockdown hypoxanthine phosphoribosyltransferase (“HPRT”). In some embodiments, the synthesized vector comprises (i) a first nucleic acid sequence to knockdown HPRT, and (ii) a second nucleic sequence encoding a therapeutic gene (including any of the therapeutic genes enumerated herein or recited above). In some embodiments, the synthesized vector comprises (i) a first nucleic acid sequence to knockdown HPRT, and (ii) a second nucleic sequence encoding a gamma-globin gene. In some embodiments, the synthesized vector comprises (i) a first nucleic acid sequence to knockdown HPRT, and (ii) a second nucleic sequence encoding a Wiskott-Aldrich Syndrome protein. In some embodiments, the synthesized vector comprises a nucleic acid sequence to knockdown CCR5.

In some embodiments, induction of viral vector production occurs in a serum-containing medium. In some embodiments, the method includes replacing the serum-containing medium between about 18 hours and about 28 hours following induction with additional serum-containing medium. In some embodiments, the method includes replacing the serum-containing medium about 24 hours following induction with additional serum-containing medium. In some embodiments, the method includes replacing the serum-containing medium between about 18 hours to about 28 hours following induction with a serum-free medium. In some embodiments, the method includes replacing the serum-containing medium about 24 hours following induction with a serum-free medium. In some embodiments, the serum-free medium may comprise lipids and/or growth factors.

In a second aspect of the present disclosure is a method of producing viral vectors from stable producer cell line cells comprising (a) synthesizing a vector by inserting one or more nucleic acid sequences into a recombinant plasmid; (b) forming a concatemeric array from an expression cassette excised from the synthesized vector and from DNA fragments obtained from an antibiotic resistance cassette plasmid; (c) transfecting one of a GPR, GPRG, GPRT, GPRGT, GPRT-G packing cell line or a derivative thereof with the formed concatemeric array to provide the stable producer cell line cells; (d) inducing viral vector production from the stable producer cell line cells; and (e) repeatedly harvesting the viral vectors in serum-free media every about 40 hours to about 56 hours following an initial harvesting of the viral vectors. In some embodiments, the initial harvesting occurs between about 40 hours to about 56 hours after induction. In some embodiments, the viral vectors are repeatedly harvested every about 44 hours to about 52 hours. In some embodiments, the viral vectors are repeatedly harvested every about 48 hours. In some embodiments, the serum-free media comprises one or more growth factors. In some embodiments, the serum-free media comprises one or more lipids. In some embodiments, the serum-free media comprises both growth factors and lipids, e.g. a mixture including one or more growth factors and/or one or more lipids. In some embodiments, the repeated harvesting comprises adding fresh serum-free media to the induced generated stable producer cell line cells without introducing additional generated stable producer cell line cells.

In some embodiments, the stable producer cell line is based on the GPRG packaging cell line. In some embodiments, the stable producer cell line is based on the GPRT packaging cell line. In some embodiments, the stable producer cell line is based on the GPR packaging cell line. In some embodiments, a ratio of the DNA fragments from the synthesized vector and the DNA fragments from the antibiotic resistance cassette ranges from about 25:1 to about 1:25. In some embodiments, the antibiotic resistance cassette plasmid is a bleomycin antibiotic resistance cassette.

In some embodiments, the recombinant plasmid comprises a nucleotide sequence having at least about 90% identity to that of SEQ ID NO: 1. In some embodiments, the recombinant plasmid comprises a nucleotide sequence having at least about 95% identity to that of SEQ ID NO: 1. In some embodiments, the recombinant plasmid comprises a nucleotide sequence having at least about 90% identity to that of SEQ ID NO: 2. In some embodiments, the recombinant plasmid comprises a nucleotide sequence having at least about 95% identity to that of SEQ ID NO: 2.

In some embodiments, the induction of viral vector production occurs in a serum-containing medium. In some embodiments, the method further comprises replacing the serum-containing media about 24 hours following induction with additional serum-containing medium. In some embodiments, the method further comprises replacing the serum-containing media about 24 hours following induction with a serum-free medium. In some embodiments, the serum-free medium may comprise lipids and/or growth factors. In some embodiments, the lipids include cholesterol, phospholipids, and fatty acids.

In some embodiments, the one or more nucleic acid sequences inserted into the recombinant plasmid is a therapeutic gene. Examples of suitable therapeutic genes are enumerated herein. In some embodiments, the one or more nucleic acid sequences inserted into the recombinant plasmid is a gamma-globin gene. In some embodiments, the one or more nucleic acid sequences inserted into the recombinant plasmid includes an RNA interference agent (“RNAi agent”) to knockdown HPRT. In some embodiments, the RNAi agent is an shRNA, a microRNA, or a hybrid thereof. In some embodiments, the one or more nucleic acid sequences inserted into the recombinant plasmid includes an RNAi to knockdown CCR5.

In some embodiments, the method provides for a production of viral titer ranging from about 0.5×10⁶ TU/mL to about 4×10⁶ TU/mL during each individual harvesting of the repeated harvesting. In some embodiments, the viral titer ranges from about 0.5×10⁶ TU/mL to about 2×10⁶ TU/mL during each individual harvesting of the repeated harvesting. In some embodiments, the viral titer ranges from about 0.5×10⁶ TU/mL to about 1.5×10⁶ TU/mL during each individual harvesting of the repeated harvesting. In some embodiments, the viral vectors are harvested at least 5 times. In some embodiments, the viral vectors are harvested at least 10 times. In some embodiments, the viral vectors are harvested at least 20 times. In some embodiments, the viral vectors are harvested at least 20 times. In some embodiments, the repeated harvesting occurs for a period of time ranging from between about 10 days to about 90 days. In some embodiments, the repeated harvesting occurs for a period of time ranging from between about 20 days to about 70 days.

In a third aspect of the present disclosure is a method of harvesting vector supernatant from stable producer cell line cells comprising: inducing viral vector production from the stable producer cell line cells; and repeatedly harvesting the viral vectors from the induced stable producer cell line cells in serum-free media every between about 40 to about 56 hours following an initial harvesting of the viral vectors. In some embodiments, the repeated harvesting comprises adding fresh serum-free media to the induced generated stable producer cell line cells without introducing additional generated stable producer cell line cells. In some embodiments, the viral vectors are first harvested about 40 hours after induction. In some embodiments, the viral vectors are repeatedly harvested every about 48 hours following the initial harvesting of the viral vectors. In some embodiments, the method provides for a production of viral titer ranging from about 0.5×10⁶ TU/mL to about 4×10⁶ TU/mL during each individual harvesting of the repeated harvesting. In some embodiments, the viral vectors are harvested at least 5 times. In some embodiments, the viral vectors are harvested at least 10 times. In some embodiments, the viral vectors are harvested at least 20 times. In some embodiments, the repeated harvesting occurs for a period of time ranging from between about 10 days to about 90 days. In some embodiments, the repeated harvesting occurs for a period of time ranging from between about 20 days to about 70 days.

In some embodiments, the serum-free media comprises one or more additives. In some embodiments, the additives include one or more growth factors. In some embodiments, the additives include one or more lipids. In some embodiments, the lipids include cholesterol, phospholipids, and fatty acids.

In some embodiments, the stable producer cell line cells are passaged in a serum-containing medium; and wherein the cells are cultured in a serum-free medium. In some embodiments, stable producer cell line cells are passaged in a serum-containing medium; and wherein the cells are cultured in serum-containing media.

In some embodiments, the viral vectors comprise a nucleic acid sequence encoding a therapeutic gene. In some embodiments, the therapeutic gene corrects for sickle cell disease or at least mitigates one symptom of a sickle cell disease. In some embodiments, the viral vector comprises a nucleic acid sequence encoding a gamma-globin gene. In some embodiments, the viral vector comprises a nucleic acid sequence to knockdown HPRT. In some embodiments, the viral vector comprises (i) a first nucleic acid sequence to knockdown HPRT, and (ii) a second nucleic sequence encoding a therapeutic gene. In some embodiments, the viral vectors comprise a nucleic acid sequence to knockdown CCR5. In some embodiments, the viral vectors comprise a nucleic acid sequence encoding CRISPR/Cas components.

In a fourth aspect of the present disclosure is a composition comprising viral vectors comprising a first nucleic acid sequence encoding an RNAi to knockdown HPPRT, wherein the viral vectors are produced by: inducing viral vector production from generated stable producer cell line cells; and repeatedly harvesting the viral vectors from the induced generated stable producer cell line cells every about 40 to about 56 hours following an initial harvesting of the viral vectors. In some embodiments, the repeated harvesting of the viral vectors comprises adding fresh media to the induced generated stable producer cell line cells without introducing additional generated stable producer cell line cells. In some embodiments, the repeated harvesting is conducted in serum-free media.

In some embodiments, the viral vectors further comprise a second nucleic acid sequence. In some embodiments, the second nucleic acid sequence encodes for a therapeutic gene, including any of those enumerated herein. In some embodiments, the therapeutic gene is a gamma globin gene. In some embodiments, the therapeutic gene is a Wiskott-Aldrich Syndrome protein. In some embodiments, the therapeutic gene is a C1 esterase inhibitor protein. In some embodiments, the therapeutic gene is a Bruton's tyrosine kinase. In some embodiments, the second nucleic acid encodes a nuclease. In some embodiments, the nuclease is selected from the group consisting of a homing endonuclease, a transcription activator-like effector nuclease, a zinc finger nuclease, Type II clustered regularly interspaced short palindromic repeats, and a megaTAL nuclease. In some embodiments, the second nucleic acid sequence encodes CRISPR/Cas components. In some embodiments, the CRISPR/Cas components are selected from the group consisting of Cas9 proteins and Cas12 proteins. In some embodiments, the viral vectors are retroviral vectors. In some embodiments, the viral vectors are lentiviral vectors.

In a fifth aspect of the present disclosure is a use of a composition comprising the viral vectors of the fourth aspect of the present disclosure in transducing host cells. Suitable host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In some embodiments, the host cell is a hematopoietic cell, such as hematopoietic progenitor/stem cell (e.g. CD34-positive hematopoietic progenitor/stem cell (HPSC)), a monocyte, a macrophage, a peripheral blood mononuclear cell, a CD4+T lymphocyte, a CD8+T lymphocyte, or a dendritic cell. In some embodiments, the host cells are rendered substantially HPRT deficient after transduction, e.g. having at least a 50% reduction in HPRT expression.

In a sixth aspect of the present disclosure is a method of repeatedly harvesting viral titer comprising: (a) a passaging phase wherein stable producer cell line cells are passaged in a serum-containing medium, (b) a culturing phase wherein the stable producer cell line cells are treated in a first serum-free medium, and (c) a production phase wherein the stable producer cell line cells are treated in a second serum-free medium. In some embodiments, the viral vectors are repeatedly harvested from induced stable producer cell line cells, where the repeated harvesting occurs in serum-free media. In some embodiments, the repeated harvesting occurs every about 40 hours to about 56 hours following an initial harvesting of the viral vectors. In some embodiments, the repeated harvesting comprises adding fresh serum-free media to the induced stable producer cell line cells without introducing additional stable producer cell line cells.

In some embodiments, the first serum-free medium comprises one or more additives. In some embodiments, the second serum-free medium comprises one or more additives. In some embodiments, the first and second serum-free mediums are the same. In some embodiments, the first and second serum-free mediums are different, e.g. each comprises a different additive component. For example, the first serum-free medium may include one type of growth factor while the second serum-free medium may include a different type of growth factor. Likewise, the first serum-free medium may include one or more growth factors while the second serum-free medium includes one or more lipids. In some embodiments, the amount of additive in any serum-free medium ranges from about 0.05% to about 10% by volume of the medium. In some embodiments, the method provides for the harvesting of viral titer in an amount ranging from between about 0.5×10⁶ TU/mL to about 4×10⁶ TU/mL during each individual harvesting of the repeated harvesting. In some embodiments, the viral vectors are harvested at least 5 times. In some embodiments, the viral vectors are harvested at least 10 times. In some embodiments, the viral vectors are harvested at least 20 times. In some embodiments, the viral vectors are harvested at least 20 times. In some embodiments, the repeated harvesting occurs for a period of time ranging from between about 10 days to about 90 days. In some embodiments, the repeated harvesting occurs for a period of time ranging from between about 20 days to about 70 days.

In some embodiments, the stable producer cell line cells are generated by (a) synthesizing a vector by cloning one or more genes into a recombinant plasmid; (b) forming a concatemeric array from (i) an expression cassette excised from the synthesized vector, and (ii) an expression cassette obtained from an antibiotic resistance cassette plasmid; (c) transfecting one of the GPR, GPRG, GPRT, GPRGT, or GPRT-G packaging cell lines with the formed concatemeric array; and (d) isolating the stable producer cell line.

In some embodiments, the synthesized vector includes a nucleic acid sequence encoding a therapeutic gene. Examples of suitable therapeutic genes are enumerated herein. In some embodiments, the therapeutic gene corrects for sickle cell disease or at least mitigates one symptom of a sickle cell disease. In some embodiments, the synthesized vector comprises a nucleic acid sequence encoding a gamma-globin gene. In some embodiments, the synthesized vector comprises a nucleic acid sequence to knockdown hypoxanthine phosphoribosyltransferase (“HPRT”). In some embodiments, the synthesized vector comprises (i) a first nucleic acid sequence to knockdown HPRT, and (ii) a second nucleic sequence encoding a therapeutic gene (e.g. a gamma-globin gene). In some embodiments, the synthesized vector comprises a nucleic acid sequence to knockdown CCR5.

In a seventh aspect of the present disclosure is a method of harvesting vector supernatant comprising: generating a stable producer cell line cells, wherein the stable producer cell line cells are derived from one of a GPR, GPRG, GPRT, GPRGT, or GPRT-G packing cell line or a derivative thereof; inducing viral vector production from the generated stable producer cell line cells; and repeatedly harvesting the viral vectors from the induced generated stable producer cell line cells in serum-free media every about 40 to about 56 hours following an initial harvesting of the viral vectors, wherein the repeated harvesting comprises adding fresh serum-free media to the induced generated stable producer cell line cells without introducing additional generated stable producer cell line cells. In some embodiments, the serum-free media comprises one or more growth factors. In some embodiments, the serum-free media comprises one or more lipids. In some embodiments, the initial harvesting of the viral vectors occurs between about 40 hours to about 56 hours after induction. In some embodiments, the initial harvesting of the viral vectors occurs less than 48 hours after induction. In some embodiments, the repeated harvesting occurs at least twice. In some embodiments, the repeated harvesting occurs every about 44 to about 52 hours. In some embodiments, the repeated harvesting occurs every about 48 hours.

In some embodiments, the serum-free media is replaced after each repeated harvesting. In some embodiments, the method provides for a production of viral titer ranging from about 0.5×10⁶ TU/mL to about 4×10⁶ TU/mL during each individual harvesting of the repeated harvesting. In some embodiments, the viral titer ranges from about 0.5×10⁶ TU/mL to about 2×10⁶ TU/mL during each individual harvesting of the repeated harvesting. In some embodiments, the viral vectors are harvested at least 5 times. In some embodiments, the viral vectors are harvested at least 10 times. In some embodiments, the viral vectors are harvested at least 20 times. In some embodiments, the repeated harvesting occurs for a period of time ranging from between about 10 days to about 90 days. In some embodiments, the repeated harvesting occurs for a period of time ranging from between about 20 days to about 70 days.

### BRIEF DESCRIPTION OF THE FIGURES

FIG. 1 illustrates that the same lentiviral vector was produced repeatedly by either transient transfection on HEK293T/17 cells according to established procedures or using a GPRG-based stable producer cell line. Vector containing media (VCM) was concentrated 100× by ultracentrifugation and lentiviral (LV) titer was determined by gene transduction assay.

FIG. 2 is a flowchart illustrating a method of generating a stable producer cell line and for harvesting lentiviral vectors produced from the generated stable producer cell line.

FIG. 3 illustrates the assessment of producer cell line stability for two different cell line MWCBs over a three-month period of continuous passage. At regular intervals, lentiviral vectors were induced by tetracycline (TET) removal and VCM was assessed for lentiviral vector titer by gene transduction assay. Both cell lines were stable and able to produce lentiviral vector in excess of 1×10⁶ TU/mL over the three-month period (about 90 days) and for in excess of about 25 passages.

FIG. 4 illustrates the kinetics of lentiviral vector production following induction by removal of TET. Vector titer was assessed in VCM by gene transduction assay. In all instances, GPRG-based stable producer cell lines were able to maintain lentiviral vector production at levels above about 1×10⁶ TU/mL (unconcentrated) for at least about 5 days following induction.

FIGS. 5A and 5B illustrate the kinetics of lentiviral vector production from stable cell lines. (FIG. 5A) During vector production, the medium was replaced with a fresh medium on a daily basis (▪) or every 2 days (□). (FIG. 5B) The total amount of lentiviral vectors in the harvested medium was titrated on 293T cells. The data shown are the mean values±SD (N=2). TUs, transduction units.

FIG. 6A illustrates GPRG and 293T cells were induced in medium without doxycycline (Dox). The induced cells were stained by anti-VSVG antibodies to detect the VSVG expression and measured by flow cytometry.

FIG. 6B illustrates the ability of the GPRG packaging cell line to produce lentiviral vector even after a prolonged culture.

FIGS. 7A and B illustrate lentiviral production in different culture conditions. (A) Cultured/Produced in serum-containing medium. (B, Left) Cultured in serum-containing/Produced in serum-free medium; (B, Right) Cultured/Produced in serum-free medium. D10: 500 mL DMEM/GlutaMAX™ (available from ThermoFisher); 50 mL FBS (10% w/v); 5 mL Pen/Strep; SFM: serum-free medium.

FIG. 8 sets forth a FACS analysis of 293T or TF-1a cells incubated with either fresh medium (no vector) or LVsh5/C46 vectors.

FIG. 9 illustrates the quantification of lentiviral vector copy numbers in the infected cells. C46 qPCR was used to determine the vector copy number per host genome after transduction at two doses (MOI=1 or 0.3).

FIG. 10 illustrates that ghost-CCR5 cells were transduced with LVsh5/C46 vectors. The decreased level of CCR5 expression was measured by FACS.

FIG. 11 sets forth a schematic diagram of pUC57-TL20.

FIG. 12 illustrates an HIV-1 based lentiviral transfer vector according to some embodiments of the present disclosure. This particular transfer vector encodes a short hairpin RNA (shRNA) for down-regulation of the HIV-1 co-receptor CCR5, in combination with a HIV-1 fusion inhibitor (C46).

FIG. 13 illustrates lentiviral induction from using the methods disclosed herein with and without serum. Cells cultured in serum-free media produced nearly as much virus as those cultured with 10% PBS. It is believed that the methods disclosed here may be adapted to serum-free culture environments.

FIG. 14 is a flowchart illustrating a method of generating DNA fragments.

FIG. 15 is a flowchart illustrating a method of synthesizing a concatemeric array.

FIG. 16 is a flowchart illustrating a method of introducing a concatemeric array into a packaging cell line.

FIG. 17 is a flowchart illustrating a method of selecting for transfected clones.

FIG. 18 is a flowchart illustrating a method of performing a single colony isolation.

FIG. 19 is a flowchart illustrating a method of evaluating viral production.

FIGS. 20A, 20B, and 20C, in general, describe producer cells for synthesizing TL20-Cal1-wpre and TL20-Unc-GFP vectors. FIG. 20A illustrates a flow cytometry analysis of 293T cells incubated with either fresh medium (left: no vector) or TL20-Cal1-WPRE (Right) harvested from the most potent producer clone. FIG. 20B illustrates a flow cytometry analysis of 293T cells incubated with either fresh medium (dark grey bar: no vector) or TL20-UbcGFP (light grey bar) harvested from the most potent producer clone. FIG. 20C illustrates the distribution of measured vector titers of supernatants from the independent producer clones for making the TL20-Cal1-WPRE (left) or TL20-UbcGFP (right) vector. The vectors were titrated on 293T cells and analyzed by flow cytometry. The highest titer achieved for the vectors prepared using polyclonal producer cells (before single clonal selection) is indicated by dashed line. Legend: Ubc: Ubiquitin C promoter; GFP: enhanced green fluorescence protein.

FIGS. 21A and 21B illustrate that the producer cell lines can generate virus in serum free media. FIG. 21A illustrates infectious titer of GFP virus during a kinetic study (continuously harvest to day-7 post-induction). FIG. 21B illustrates transduction efficiency of the LVsh5/C46 vector (harvest on day-3 post-induction).

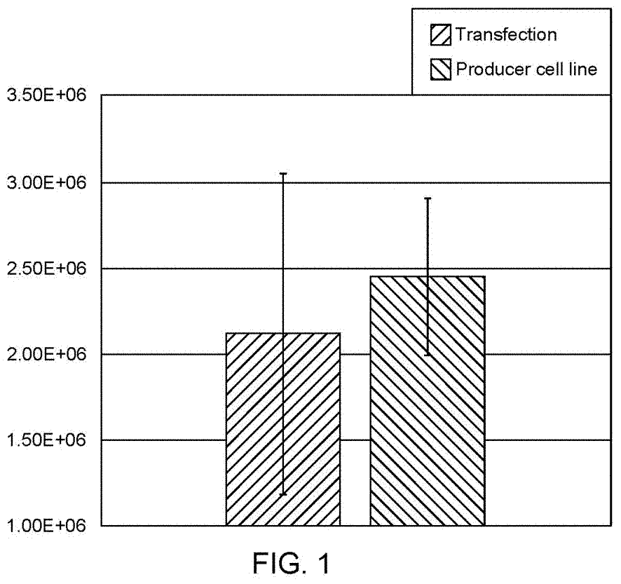

### SEQUENCE LISTING

The nucleic and amino acid sequences provided herein are shown using standard letter abbreviations for nucleotide bases, and three letter code for amino acids, as defined in 37 C.F.R. 1.822. The sequence listing is submitted as an ASCII text file, named “2016-05-09 Cal-0013WO_ST25.txt” created on May 9, 2016, 5 KB, which is incorporated by reference herein.

### DETAILED DESCRIPTION

### Definitions

As used herein, the singular terms “a,” “an,” and “the” include plural referents unless the context clearly indicates otherwise. Similarly, the word “or” is intended to include “and” unless the context clearly indicates otherwise.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “cloning” refers to the process of ligating a nucleic acid molecule into a plasmid and transferring it into an appropriate host cell for duplication during propagation of the host.

As used herein, the term “fragment” refers to polypeptides and is defined as any discrete portion of a given polypeptide that is unique to or characteristic of that polypeptide. The term as used herein also refers to any discrete portion of a given polypeptide that retains at least a fraction of the activity of the full-length polypeptide.

As used herein, the term “gene” refers to any nucleotide sequence, DNA or RNA, at least some portion of which encodes a discrete final product, typically, but not limited to, a polypeptide, which functions in some aspect of a cellular process. The term is not meant to refer only to the coding sequence that encodes the polypeptide or other discrete final product but may also encompass regions preceding and following the coding sequence that modulate the basal level of expression, as well as intervening sequences (“introns”) between individual coding segments (“exons”). In some embodiments, a gene may include regulatory sequences (e.g., promoters, enhancers, polyadenylation sequences, termination sequences, Kozak sequences, TATA box, etc.) and/or modification sequences. In some embodiments, a gene may include references to nucleic acids that do not encode proteins but rather encode functional RNA molecules such as tRNAs, RNAi-inducing agents, etc.

As used herein, the term “HIV” includes not only HIV-1, but also the various strains of HIV-1 (e.g. strain BaL or strain SF162) and the various subtypes of HIV-1 (e.g. subtypes A, B, C, D, F, G H, J, and K).

As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

As used herein, the term “lentivirus” refers to a genus of retroviruses that is capable of infecting dividing and non-dividing cells. Several examples of lentiviruses include HIV (human immunodeficiency virus: including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (Hy), which causes immune deficiency in cats; bovine immune deficiency virus (BIV), which causes lymphadenopathy, lymphocytosis, and possibly central nervous system infection in cattle; and simian immunodeficiency virus (SIV), which causes immune deficiency and encephalopathy in sub-human primates.

As used herein, the term “lentiviral vector” is used to denote any form of a nucleic acid derived from a lentivirus and used to transfer genetic material into a cell via transduction. The term encompasses lentiviral vector nucleic acids, such as DNA and RNA, encapsulated forms of these nucleic acids, and viral particles in which the viral vector nucleic acids have been packaged.

As used herein, the term “multiple cloning site” or “MCS” refers to nucleotide sequences comprising restriction sites for the purpose of cloning nucleic acid fragments into a cloning vector plasmid. An MCS, also referred to as a polylinker or polycloning site, is a cluster of cloning sites such that many restriction enzymes are able to operate within the site. A cloning site in some embodiments is a known sequence upon which a restriction enzyme operates to linearize or cut a plasmid.

As used herein, the term “producer cell” refers to a cell which contains all the elements necessary for production of lentiviral vector particles.

As used herein, the term “packaging cell” refers to a cell which contains those elements necessary for production of infectious recombinant virus which are lacking in a recombinant viral vector, retroviral vector, or lentiviral transfer vector plasmid. Typically, such packaging cells contain one or more expression cassettes which are capable of expressing viral structural proteins (such as gag, pol and env) but they do not contain a packaging signal. Packaging cell line cells are typically mammalian cell line, which contains the necessary coding sequences to produce viral particles which lack the ability to package RNA and produce replication-competent helper-virus. When the packaging function is provided within the cell line (e.g., in trans), the packaging cell line produces recombinant retrovirus (or lentivirus), thereby becoming a “producer cell line.”

As used herein, the terms “restriction endonuclease” or “restriction enzyme” refer to a member or members of a class of catalytic molecules that bind a cognate sequence of a nucleic acid molecule (e.g. DNA) and cleave it at a precise location within that sequence.

As used herein, the term “retrovirus” refers to viruses having an RNA genome that is reverse transcribed by retroviral reverse transcriptase to a cDNA copy that is integrated into the host cell genome. Retroviral vectors and methods of making retroviral vectors are known in the art. Briefly, to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., Cell, Vol. 33:153-159, 1983). When a recombinant plasmid containing a cDNA, together with the retroviral LTR and packaging sequences, is introduced into this cell line, the packaging sequence allows the RNA transcript of the recombinant plasmid to be packaged into viral particles, which are then secreted into the culture media. The media containing the recombinant retroviruses is then collected, optionally concentrated, and used for gene transfer. In some embodiments, term “retrovirus” refers to any known retrovirus (e.g., type c retroviruses, such as Moloney murine leukemia virus (MoMuLV), Harvey murine sarcoma virus (HaMuSV), murine mammary tumor virus (MuMTV), gibbon ape leukemia virus (GaLV), feline leukemia virus (FLV) and Rous Sarcoma Virus (RSV)). “Retroviruses” of the invention also include human T cell leukemia viruses, HTLV-1 and HTLV-2, and the lentiviral family of retroviruses, such as human Immunodeficiency viruses, HIV-1, HIV-2, simian immunodeficiency virus (SIV), feline immunodeficiency virus (FIV), equine immunodeficiency virus (EIV), and other classes of retroviruses.

As used herein, “RNA interference agents” or “RNAi agents” are inhibitory or silencing nucleic acids. As used herein, a “silencing nucleic acid” refers to any polynucleotide which is capable of interacting with a specific sequence to inhibit gene expression. Examples of silencing nucleic acids include RNA duplexes (e.g. siRNA, shRNA), locked nucleic acids (“LNAs”), antisense RNA, DNA polynucleotides which encode sense and/or antisense sequences of the siRNA or shRNA, DNAzymses, or ribozymes. The skilled artisan will appreciate that the inhibition of gene expression need not necessarily be gene expression from a specific enumerated sequence, and may be, for example, gene expression from a sequence controlled by that specific sequence.

As used herein, the term “seeding” refers to the process of providing a cell culture to a bioreactor or another vessel for cell or vector culture production.

As used herein, the phrases “serum-free media” or “serum-free medium” refer to a media which contains no serum, i.e. a cell culture medium that does not contain sera from animal or human origin. In some embodiments, a serum-free medium is protein free and also free from hydrolysates or components of unknown composition. Suitable cell culture media are known to the person skilled in the art. These media may optionally comprise salts, vitamins, buffers, energy sources, amino acids and other substances. An example of a medium suitable for the serum free cultivation of cells is medium 199 (Morgan, Morton and Parker; Proc. Soc. Exp. Biol. Med. 1950, 73, 1; obtainable inter alia from 10 Life Technologies).

As used herein, the term “shRNA” refers to RNA molecules comprising an antisense region, a loop portion and a sense region, wherein the sense region has complementary nucleotides that base pair with the antisense region to form a duplex stem. Following post-transcriptional

As used herein, the term “therapeutic gene” refers to a gene that can be administered to a subject for the purpose of treating or preventing a disease. Encompassed within the definition of “therapeutic gene” is a “biologically functional equivalent” therapeutic gene. As will be understood by those in the art, the term “therapeutic gene” includes genomic sequences, cDNA sequences, and smaller engineered gene segments that express, or may be adapted to express, proteins, polypeptides, domains, fusion proteins, and mutants that maintain some or all of the therapeutic function of the full-length polypeptide encoded by the therapeutic gene. Accordingly, sequences that have about 70% sequence homology to about 99% sequence homology and any range or amount of sequence homology derivable therein, such as, for example, about 70% to about 80%, and more preferably about 85% and about 90%; or even more preferably, between about 95% and about 99%; of amino acids that are identical or functionally equivalent to the amino acids of the therapeutic gene will be sequences that are biologically functional equivalents provided the biological activity of the polypeptide is maintained.

As used herein, the terms “transduce,” or “transduction” refer to the delivery of a gene(s) using a viral or retroviral vector by means of infection rather than by transfection. For example, an anti-HPRT gene carried by a retroviral vector (a modified retrovirus used as a vector for introduction of nucleic acid into cells) can be transduced into a cell through infection and provirus integration. Thus, a “transduced gene” is a gene that has been introduced into the cell via lentiviral or vector infection and provirus integration. Viral vectors (e.g., “transducing vectors”) transduce genes into “target cells” or host cells.

As used herein, the term “vector” refers to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication or may include sequences sufficient to allow integration into host cell DNA. As will be evident to one of ordinary skill in the art, viral vectors may include various viral components in addition to nucleic acid(s) that mediate entry of the transferred nucleic acid. Numerous vectors are known in the art including, but not limited to, linear polynucleotides, polynucleotides associated with ionic or amphiphilic compounds, plasmids, and viral vectors. Examples of viral vectors include, but are not limited to, adenoviral vectors, adeno-associated virus vectors, retroviral vectors (including lentiviral vectors), and the like.

As used herein, the term “vector copy number” or “VCN” refers to the number of copies of a vector, or portion thereof, in a cell's genome. The average VCN may be determined from a population of cells or from individual cell colonies. Exemplary methods for determining VCN include polymerase chain reaction (PCR) and flow cytometry.

As used herein, the terms “viral titer” or “titer,” used interchangeably herein, refer to the number of infectious viral particles in a sample of body fluid (e.g., blood, serum, plasma, saliva, urine) from an infected individual.

A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. See, for example, “RNA Viruses: A Practical Approach” (Alan J. Cann, Ed., Oxford University Press, (2000)); 0 Narayan and Clements (1989) J. Gen. Virology, Vol. 70:1617-1639; Fields et al. (1990) Fundamental Virology Raven Press.; Miyoshi H, Blamer U, Takahashi M, Gage F H, Verma I M. (1998) J Virol., Vol. 72(10):8150 7, and U.S. Pat. No. 6,013,516.

Lentiviral vectors are known in the art, including several that have been used to infect hematopoietic progenitor/stem cells (HPSC). Such vectors can be found, for example, in the following publications, which are incorporated herein by reference: Evans et al., Hum Gene Ther., Vol. 10:1479-1489, 1999; Case et al., Proc Natl Acad Sci USA, Vol. 96:2988-2993, 1999; Uchida et al., Proc Natl Acad Sci USA, Vol. 95:11939-11944, 1998; Miyoshi et al., Science, Vol. 283:682-686, 1999; and Sutton et al., J. Virol., Vol. 72:5781-5788, 1998. In one embodiment, the expression vector is a modified lentivirus, and thus is able to infect both dividing and non-dividing cells. Further, the modified lentiviral genome preferably lacks genes for lentiviral proteins required for viral replication, thus preventing undesired replication, such as replication in the target cells. The required proteins for replication of the modified genome are preferably provided in trans in the packaging cell line during production of the recombinant retrovirus (or specifically lentivirus). In one embodiment, the packaging cell line is a 293T cell line. The lentiviral vector preferably comprises sequences from the 5′ and 3′ long terminal repeats (LTRs) of a lentivirus. In one embodiment, the viral construct comprises the R and U5 sequences from the 5′ LTR of a lentivirus and an inactivated or self-inactivating 3′ LTR from a lentivirus. The LTR sequences may be LTR sequences from any lentivirus including from any species or strain. For example, the LTR may be LTR sequences from HIV, simian immunodeficiency virus (SIV), feline immunodeficiency virus (FIV) or bovine immunodeficiency virus (BIV). Preferably the LTR sequences are HIV LTR sequences.

Method of Generating Stable Producer Cell Line Cells

Lentiviral vectors (LVs) are important tools for gene transfer due to their efficiency and ability to stably transduce both dividing and non-dividing cells. As a result, investigators are using them as gene delivery vehicles in a wide variety of clinical applications. Nevertheless, large-scale clinical production using current good manufacturing practice (cGMP) methods comes with a set of challenges that must be considered as more clinical trials using lentiviral vectors receive regulatory approval. One important consideration in designing cGMP-compatible processes is the need to integrate regulatory considerations into manufacturing processes that are capable of producing consistent lentivirus for multiple cGMP productions. The vast majority of lentiviral vectors being used clinically has been produced by transient transfection. Transient transfection-based production is, however, often labor intensive and subject to variation. For this reason, several stable packaging cell line systems have recently been developed. While the use of these cell lines for the bio-manufacturing of retroviral and lentiviral vectors is particularly attractive for both scalability and consistency, development of such lines is time consuming and the regulatory pathway for the cGMP use of these lines has not been firmly established.

In view of the foregoing, the present disclosure sets forth a process for the clinical production of retroviral vectors, including self-inactivating lentiviral vectors (SIN-LVs). It is believed that through the use of a novel lentiviral transfer vector plasmid together with packaging cell lines (e.g. GPR, GPRG, GPRT, GPRGT or GPRT-G or derivative or analog packaging cell line derived therefrom), that stable producer cell line cells may be generated so as to enable the production of retroviral vectors, including self-inactivating lentiviral vectors (e.g. LVgGsh7; a vector comprising a component designed to knockdown expression of hypoxanthine-guanine phosphoribosyltransferase (HPRT); a vector comprising a first component designed to knockdown HPRT expression and also including a second component for the expression of a therapeutic gene, such as a gamma-globin gene). While certain embodiments and examples described herein refer to the production of LVsh5/C46, which is a self-inactivating lentiviral vector encoding a short hairpin RNA (shRNA) for down-regulation of the HIV-1 co-receptor CCR5, in combination with a HIV-1 fusion inhibitor (namely, C46), the skilled artisan will recognize that the methods described herein are suitable for the generation of stable producer cell line cells capable of producing any SIN-LVs, comprising any desired or client supplied genes or sequences (e.g. a SIN-LIV designed to express a nucleic acid sequence encoding a gamma-globin gene, such as those nucleic acid sequences disclosed in US Patent Application Publication No. 2017/0145077, the disclosure of which is hereby incorporated by reference herein in its entirety; or a SIN-LIV designed to express a nucleic acid sequence encoding a Wiskott-Aldrich syndrome protein).

Applicant has demonstrated that compared to SIN-LVs produced by transient transfection, that the presently disclosed method (i) is capable of generating a similar quality and quantity of SIN-LVs; (ii) produces SIN-LVs that may have better potency; and (ii) enables a process which maintains yields while greatly decreasing the prep-to-prep variability seen with transient transfection.

pUC57-TL20c

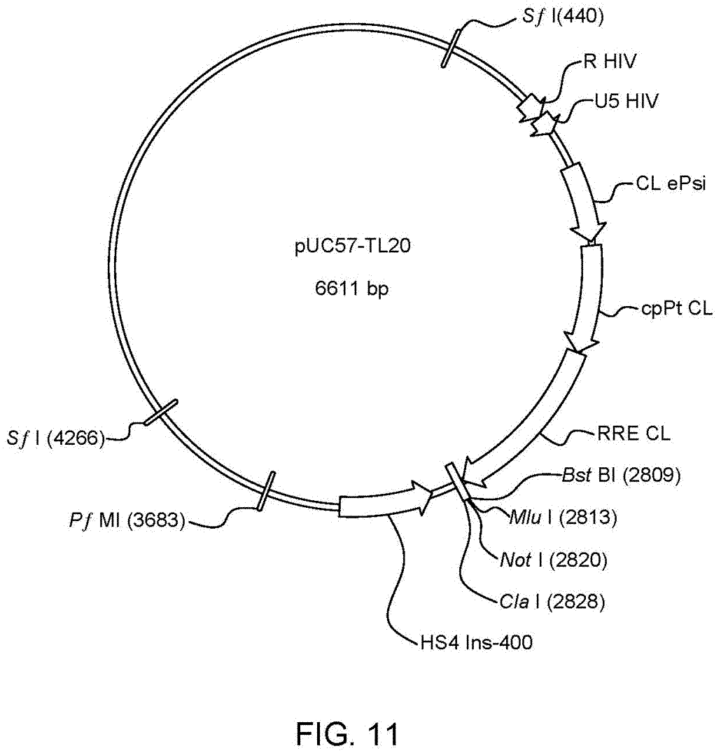

The present disclosure provides, in some embodiments, a human immunodeficiency virus type 1 (HIV-1) based third generation, self-inactivating (SIN) lentiviral transfer vector plasmid (hereinafter referred to as “pUC57-TL20”) comprising a novel, versatile multiple cloning site (MC S) (see FIG. 11).

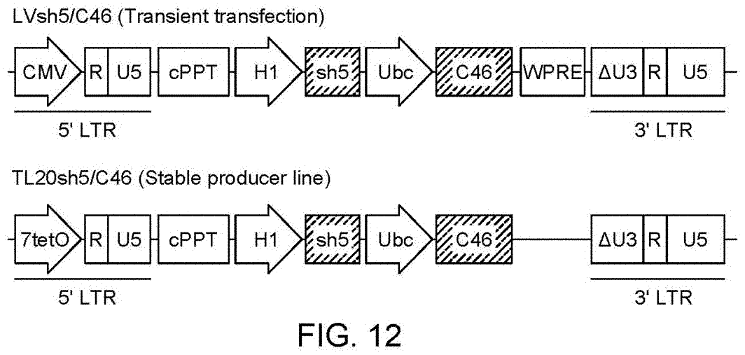

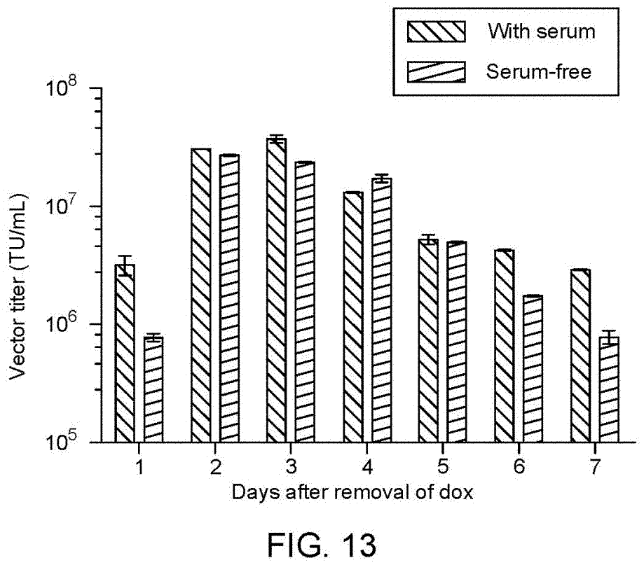

In some embodiments, the lentiviral vector transfer plasmid comprises a vector backbone (“TL20c”) that does not itself comprise an internal promoter (hence, it is “promoterless”). In some embodiments, the lentiviral vector transfer plasmid comprises one promoter, e.g. a tetracycline repressible promoter, upstream of the vector backbone (see FIG. 12). Without wishing to be bound by any particular theory, it is believed that the promoterless design of the vector backbone allows for the generation of a lentiviral transfer vector plasmid that enables the delivery and subsequent expression of a gene of interest from a user-determined promoter.

FIG. 11 sets forth a gene map illustrating the constituent elements of the lentiviral vector transfer plasmid. In some embodiments, the lentiviral vector transfer plasmid comprises between about 6500 nucleotides and about 6750 nucleotides. In other embodiments, the lentiviral vector transfer plasmid comprises between 6600 nucleotides and about 6700 nucleotides. In some embodiments, the vector backbone of the lentiviral transfer vector plasmid comprises between about 3850 nucleotides and about 3950 nucleotides. In some embodiments, the vector backbone of the lentiviral transfer vector plasmid comprises about 3901 nucleotides.

As shown in FIG. 11, the plasmid comprises a 5′ flanking HIV LTR, a packaging signal or ψ+, a central polypurine tract (cPPT), a Rev-response element (RRE), a multiple cloning site (MCS), and a 3′ flanking HIV LTR. The LTR regions further comprise a U3 and U5 region, as well as an R region.

According to certain embodiments of the disclosure, the transfer plasmid includes a self-inactivating (SIN) LTR. As is known in the art, during the retroviral life cycle, the U3 region of the 3′ LTR is duplicated to form the corresponding region of the 5′ LTR in the course of reverse transcription and viral DNA synthesis. Creation of a SIN LTR is achieved by inactivating the U3 region of the 3′ LTR (preferably by deletion of a portion thereof, e.g. removal of a TATA sequence). The alteration is transferred to the 5′ LTR after reverse transcription, thus eliminating the transcriptional unit of the LTRs in the provirus, which is believed to prevent mobilization by replication competent virus. An additional safety enhancement is provided by replacing the U3 region of the 5′ LTR with a heterologous promoter to drive transcription of the viral genome during production of viral particles.

In some embodiments, the packaging signal comprises about 361 base pairs of the Gag sequence and about 448 base pairs of the Pol sequence of wild-type HIV (e.g. HIVO1 HXB2_LAI_IIIB). In some embodiments, the cPPT comprises about 85 base pairs of the Vif sequence of wild-type HIV. In some embodiments, a HIV Polypurine tract (pPu) comprises about 106 base pairs of the Nef sequence of wild-type HIV. In some embodiments, the RRE comprises about 26 base pairs of the Rev sequence, about 25 base pairs of the tat sequence, and about 769 base pairs of the Env sequence of wild-type HIV. In some embodiments, the transfer plasmid comprises a chromatin insulator and/or a beta-globulin polyadenylation signal.

In some embodiments, the nucleotide sequence encoding the packing signal comprises the sequence of SEQ ID NO: 3, or a sequence having at least 85% identity to that of SEQ ID NO: 3. In some embodiments, the nucleotide sequence encoding the packing signal comprises the sequence of SEQ ID NO: 3, or a sequence having at least 90% identity to that of SEQ ID NO: 3. In some embodiments, the nucleotide sequence encoding the packing signal comprises the sequence of SEQ ID NO: 3, or a sequence having at least 95% identity to that of SEQ ID NO: 3.

In some embodiments, the nucleotide sequence encoding the central polypurine tract (cPPT) comprises the sequence of SEQ ID NO: 4, or a sequence having at least 85% identity to that of SEQ ID NO: 4. In some embodiments, the nucleotide sequence encoding the central polypurine tract (cPPT) comprises the sequence of SEQ ID NO: 4, or a sequence having at least 90% identity to that of SEQ ID NO: 4. In some embodiments, the nucleotide sequence encoding the central polypurine tract (cPPT) comprises the sequence of SEQ ID NO: 4, or a sequence having at least 95% identity to that of SEQ ID NO: 4.

In some embodiments, the nucleotide sequence encoding the Rev response element comprises the sequence of SEQ ID NO: 5, or a sequence having at least 85% identity to that of SEQ ID NO: 5. In some embodiments, the nucleotide sequence encoding the Rev response element comprises the sequence of SEQ ID NO: 5, or a sequence having at least 90% identity to that of SEQ ID NO: 5. In some embodiments, the nucleotide sequence encoding the Rev response element comprises the sequence of SEQ ID NO: 5, or a sequence having at least 95% identity to that of SEQ ID NO: 5.

In some embodiments, the nucleotide sequence encoding the self-inactivating long terminal repeat comprises the sequence of SEQ ID NO: 6, or a sequence having at least 85% identity to that of SEQ ID NO: 6. In some embodiments, the nucleotide sequence encoding the self-inactivating long terminal repeat comprises the sequence of SEQ ID NO: 6, or a sequence having at least 90% identity to that of SEQ ID NO: 6. In some embodiments, the nucleotide sequence encoding the self-inactivating long terminal repeat comprises the sequence of SEQ ID NO: 6, or a sequence having at least 95% identity to that of SEQ ID NO: 6.

In some embodiments, the plasmid comprises a nucleotide sequence encoding a doxycycline repressible promoter that has at least 85% identity to that of SEQ ID NO: 10. In some embodiments, the plasmid comprises a nucleotide sequence encoding a doxycycline repressible promoter that has at least 90% identity to that of SEQ ID NO: 10. In some embodiments, the plasmid comprises a nucleotide sequence encoding a doxycycline repressible promoter that has at least 95% identity to that of SEQ ID NO: 10.

In some embodiments, the plasmid comprises a nucleotide sequence encoding an HIV LTR R5 region that has at least 85% identity to that of SEQ ID NO: 11. In some embodiments, the plasmid comprises a nucleotide sequence encoding an HIV LTR R5 region that has at least 90% identity to that of SEQ ID NO: 11. In some embodiments, the plasmid comprises a nucleotide sequence encoding an HIV LTR R5 region that has at least 95% identity to that of SEQ ID NO: 11.

In some embodiments, the plasmid comprises a nucleotide sequence encoding an HIV LTR U5 region that has at least 85% identity to that of SEQ ID NO: 12. In some embodiments, the plasmid comprises a nucleotide sequence encoding an HIV LTR U5 region that has at least 90% identity to that of SEQ ID NO: 12. In some embodiments, the plasmid comprises a nucleotide sequence encoding an HIV LTR U5 region that has at least 95% identity to that of SEQ ID NO: 12.

In some embodiments, the plasmid comprises a nucleotide sequence encoding a chromatin insulator that has at least 85% identity to that of SEQ ID NO: 13 In some embodiments, the plasmid comprises a nucleotide sequence encoding a chromatin insulator that has at least 90% identity to that of SEQ ID NO: 13. In some embodiments, the plasmid comprises a nucleotide sequence encoding a chromatin insulator that has at least 95% identity to that of SEQ ID NO: 13.

In some embodiments, the plasmid comprises a nucleotide sequence encoding a beta-globin polyadenylation signal that has at least 85% identity to that of SEQ ID NO: 14. In some embodiments, the plasmid comprises a nucleotide sequence encoding a beta-globin polyadenylation signal that has at least 90% identity to that of SEQ ID NO: 14. In some embodiments, the plasmid comprises a nucleotide sequence encoding a beta-globin polyadenylation signal that has at least 95% identity to that of SEQ ID NO: 14.

In some embodiments, the plasmid comprises a nucleotide sequence that has at least 85% identity to that of SEQ ID NO: 15. In some embodiments, the plasmid comprises a nucleotide sequence that has at least 90% identity to that of SEQ ID NO: 15. In some embodiments, the plasmid comprises a nucleotide sequence that has at least 95% identity to that of SEQ ID NO: 15.

The disclosure provides lentiviral transfer vector plasmids incorporating an MCS for a variety of different restriction enzymes. According to certain embodiments of the disclosure, the MCS comprises a sequence having between about 20 and 40 nucleotides. In some embodiments, the MCS of the presently disclosed plasmid comprises at least two restriction enzyme cutting sites. In other embodiments, the MCS of the presently disclosed plasmid comprises at least three restriction enzyme cutting sites. In other embodiments, the MCS of the presently disclosed plasmid comprises at least four restriction enzyme cutting sites. In yet other embodiments, the MCS of the presently disclosed plasmid comprises between about 2 and about 10 restriction sites. In yet further embodiments, the MCS of the presently disclosed plasmid comprises between about 3 and about 8 restriction sites. In some embodiments, the restriction sites within the MCS are selected from BstBI, MluI, NotI, ClaI, ApaI, XhoI, XbaI, HpaI, NheI, PacI, NsiI, SphI, Sma/Xma, AccI, BamHI, and SphI, or any derivatives or analog thereof.

In some embodiments, the MCS region of the lentiviral transfer vector plasmid carries four unique restriction enzyme cutting sites which are believed to facilitate easy sub-cloning of a desired transgene cassette. In some embodiments, the multiple cloning site comprises the BstBI, MluI, NotI, and ClaI restriction endonuclease sites. In some embodiments, the nucleotide sequence encoding the multiple cloning site comprises the sequence of SEQ ID NO: 7, or a sequence having at least 90% identity to that of SEQ ID NO: 7. There restriction site may be arranged in any order.

In some embodiments, the transfer plasmid comprises one or more additional restriction enzyme cutting sites flanking the vector backbone (see FIG. 11). In some embodiments, the transfer plasmid comprises two additional restriction enzyme cutting sites flanking the vector backbone. Without wishing to be bound by any particular theory, it is believed that the additional flanking restriction enzyme cutting sites allow for the generation of a directional (a “head-to-tail”) concatemeric array. In some embodiments, the restriction enzyme cutting sites are selected from Sfil and Bsu36I. In some embodiments, a lentiviral vector comprising one or more genes is derived from the plasmid.

In some embodiments, the lentiviral vector transfer plasmid comprises a nucleotide sequence having at least 80% identity to that of sequence of SEQ ID NO: 1. In other embodiments, the lentiviral vector transfer plasmid comprises a nucleotide sequence having at least 85% identity to that of sequence of SEQ ID NO: 1. In yet other embodiments, the lentiviral vector transfer plasmid comprises a nucleotide sequence having at least 90% identity to that of sequence of SEQ ID NO: 1. In further embodiments, the lentiviral vector transfer plasmid comprises a nucleotide sequence having at least 95% identity to that of sequence of SEQ ID NO: 1. In further embodiments, the lentiviral vector transfer plasmid comprises a nucleotide sequence having at least 96% identity to that of sequence of SEQ ID NO: 1. In yet further embodiments, the lentiviral vector transfer plasmid comprises a nucleotide sequence having at least 97% identity to that of sequence of SEQ ID NO: 1. In yet further embodiments, the lentiviral vector transfer plasmid comprises a nucleotide sequence having at least 98% identity to that of sequence of SEQ ID NO: 1. In yet further embodiments, the lentiviral vector transfer plasmid comprises a nucleotide sequence having at least 99% identity to that of sequence of SEQ ID NO: 1. In some embodiments, the lentiviral vector transfer plasmid comprises the sequence of SEQ ID NO: 1. In some embodiments, the lentiviral vector transfer plasmid has a sequence that differs by not more than 100 nucleotides from the sequence set forth in SEQ ID NO: 1.

In some embodiments, the lentiviral transfer vector plasmid comprises a nucleotide sequence having at least 80% identity to that of sequence of SEQ ID NO: 2. In other embodiments, the lentiviral vector transfer plasmid comprises a nucleotide sequence having at least 85% identity to that of sequence of SEQ ID NO: 2. In yet other embodiments, the lentiviral vector transfer plasmid comprises a nucleotide sequence having at least 90% identity to that of sequence of SEQ ID NO: 2. In further embodiments, the lentiviral vector transfer plasmid comprises a nucleotide sequence having at least 95% identity to that of sequence of SEQ ID NO: 2. In further embodiments, the lentiviral vector transfer plasmid comprises a nucleotide sequence having at least 96% identity to that of sequence of SEQ ID NO: 2. In yet further embodiments, the lentiviral vector transfer plasmid comprises a nucleotide sequence having at least 97% identity to that of sequence of SEQ ID NO: 2. In yet further embodiments, the lentiviral vector transfer plasmid comprises a nucleotide sequence having at least 98% identity to that of sequence of SEQ ID NO: 2. In yet further embodiments, the lentiviral vector transfer plasmid comprises a nucleotide sequence having at least 99% identity to that of sequence of SEQ ID NO: 2. In some embodiments, the lentiviral vector transfer plasmid comprises the sequence of SEQ ID NO: 2. In some embodiments, the lentiviral vector transfer plasmid has a sequence that differs by not more than 100 nucleotides from the sequence set forth in SEQ ID NO: 2.

In some embodiments, the lentiviral transfer vector plasmid is synthesized according to those methods known to those of skill in the art. For example, the plasmids may be synthesized using traditional restriction digestion and ligation techniques known to those of ordinary skill in the art. For example, a donor plasmid comprising the TL20c vector backbone may be subcloned into a pU57C recipient plasmid (e.g. such as those available commercially from Genescript), using standard digestion and ligation procedures known to those of ordinary skill in the art (see, for example, Sambrook et al. (1989) Molecular Cloning: A Laboratory Manual, 2nd Ed. Cold Spring Harbor, N.Y., the disclosure of which is hereby incorporated by reference herein in its entirety).

The present disclosure also includes a method of producing a retroviral vector, including a lentiviral vector, e.g. LVgGsh7 which is a lentiviral vector including a component designed to knockdown HPRT, or a lentiviral vector including a first component designed to knockdown HPRT and a second component encoding a therapeutic gene, such as a gamma globin gene (see, for example, the vector sand nucleic acid sequences disclosed in WO/2019/018383, the disclosure of which is hereby incorporated by reference herein in its entirety). In some embodiments, the method comprises synthesizing a cDNA of a gene (including any of the genes disclosed herein) and cloning the synthesized cDNA into a restriction site of a recombinant plasmid, such as pUC57-TL20c. Any therapeutic gene may be inserted into an appropriate cloning site using techniques known to those of skill in the art. In some embodiments, a gene may be amplified by polymerase chain reaction (“PCR”) and then cloned into a recombinant plasmid including a desired promoter or gene-expression controlling element (examples of suitable promoters are disclosed within WO/2019/018383, the disclosure of which is hereby incorporated by reference herein in its entirety).

In some embodiments, and solely by way of example, the method comprises synthesizing a cDNA of a gene which expresses a protein capable of preventing HIV fusion into a cell or HIV replication; and then cloning the synthesized cDNA into a restriction site in a plasmid as disclosed herein.

Generation of Stable Producer Cell Line Cells and the Repeated Harvesting of Lentiviral Vectors Produced Therefrom

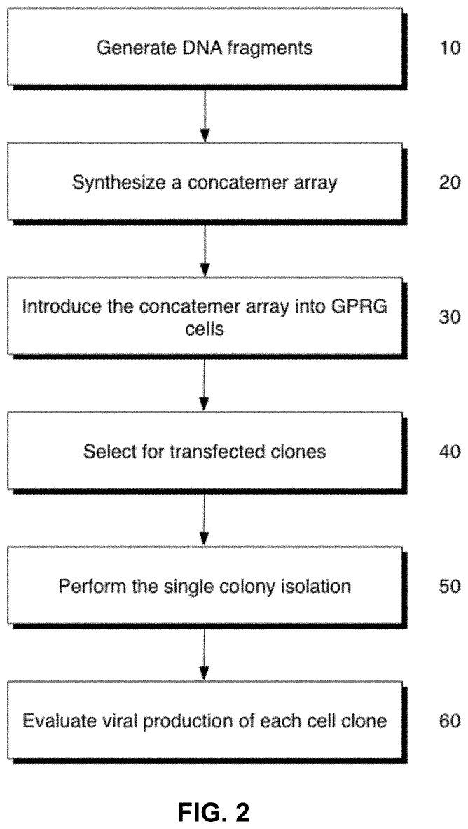

In some embodiments of the present disclosure are methods of forming stable producer cell line cells and harvesting retroviral vectors (including lentiviral vectors) produced from the generated stable producer cell line cells. In some embodiments, the lentiviral vector produced from the generated producer cell line cells are repeatedly harvested, such as repeatedly harvested every about 40 to about 56 hours. With reference to FIG. 2, a first step in producing a stable producer cell line includes generating DNA fragments (10) from first and second plasmids, where one of the plasmids is an antibiotic resistance cassette plasmid. For example, the DNA fragments may be generated from a lentiviral vector transfer plasmid and an antibiotic resistance cassette plasmid. Following DNA fragment generation (10), the DNA is then used to form a concatemeric array (20).

Subsequently, the concatemeric array is then introduced, such as by transfection, into a packing cell line (30) (e.g. GPR, GPRG, GPRT, GPRG, GPRT-G or derivatives thereof packaging cell lines). Following introduction of the array (30) and subsequent transfection, clones are selected (40) and isolated (50) to generate the stable producer cell line (60). Vector supernatant comprising lentiviral vector may then be harvested, e.g. repeatedly harvested every about 40 to about 56 hours in serum-free media.

Concatemeric Array Formation and Purification

A “concatemer” or “concatemeric array,” used interchangeably herein, refers to a long continuous DNA molecule that contains multiple copies of the same DNA sequences linked directly or indirectly in series. In some embodiments, the concatemeric array is generated and used in the transfection of the packaging cell line cells. In some embodiments, the concatemeric arrays are large arrays of linked vector genome expression cassettes, with antibiotic resistance cassettes interspersed therein.

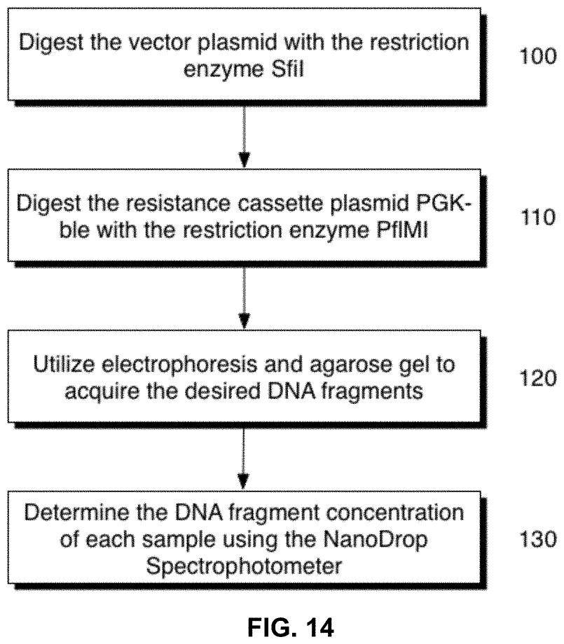

With reference to FIG. 14, DNA fragments from a lentiviral transfer vector plasmid (step 100) and an antibiotic resistance cassette plasmid are generated (step 110) to form the concatemeric array. In some embodiments, the DNA fragments may be prepared by digesting each of the plasmids according to protocols known to those of ordinary skill in the art and then ligating the digested fragments. In some embodiments, electrophoresis and agarose gel are utilized to acquire the desired DNA fragments (step 120). In some embodiments, a DNA fragment concentration may be determined using a NanoDrop Spectrophotometer (step 130). A variety of strategies are available for ligating fragments of DNA, the choice of which depends on the nature of the termini of the DNA fragments and which choices can be readily made by the skilled artisan.

In some embodiments, the lentiviral transfer vector plasmid is based on pUC57-TL20c. In some embodiments, the antibiotic resistance cassette plasmid is driven by the PGK promoter. In some embodiments, the antibiotic resistance cassette plasmid comprises flanking sites for concatemerization with the lentivirus cassette in the lentiviral transfer vector plasmid. In some embodiments, the antibiotic resistance cassette plasmid is PGK-ble (bleomycin resistance). In some embodiments, the PGK-ble plasmid comprises a nucleotide sequence having at least 90% identity to the sequence of SEQ ID NO: 9. In other embodiments, the PGK-ble plasmid comprises a nucleotide sequence having at least 95% identity to the sequence of SEQ ID NO: 9. In other embodiments, the PGK-ble plasmid has the nucleotide sequence of SEQ ID NO: 9. In some embodiments, the concatemeric arrays are formed through the in vitro ligation of generated DNA fragments derived from the lentiviral transfer vector plasmid and the PGK-ble plasmid.

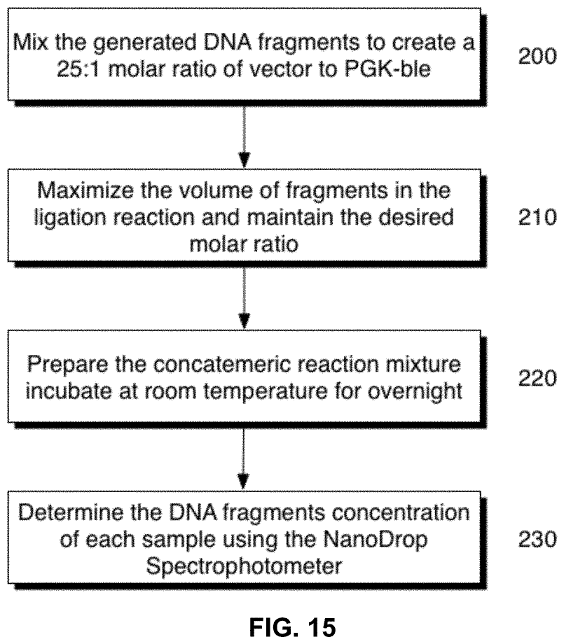

FIG. 15 outlines the general steps used in forming the concatemeric array. At step 200, generated DNA fragments are mixed and the volume of fragments in the ligation reaction are maximized to maintain the desired ratio (step 210). In some embodiments, a ratio of an amount of lentiviral transfer vector plasmid DNA to an amount of antibiotic resistance cassette plasmid DNA ranges from about 100:1 to about 1:100. In some embodiments, a ratio of an amount of lentiviral transfer vector plasmid DNA to an amount of antibiotic resistance cassette plasmid DNA ranges from about 75:1 to about 1:75. In other embodiments, a ratio of an amount of lentiviral transfer vector plasmid DNA to an amount of antibiotic resistance cassette plasmid DNA ranges from about 50:1 to about 1:50. In yet other embodiments, a ratio of an amount of lentiviral transfer vector plasmid DNA to an amount of antibiotic resistance cassette plasmid DNA ranges from about 25:1 to about 1:25. In further embodiments, a ratio of an amount of lentiviral transfer vector plasmid DNA to an amount of antibiotic resistance cassette plasmid DNA ranges from about 10:1 to about 1:10.

In some embodiments, the concatemeric reaction mixture is incubated overnight at room temperature (step 220), e.g. at a temperature ranging from between about 20° C. to about 25° C. Subsequently, the DNA fragment concentration for each sample may then be measured using a NanoDrop Spectrophotometer (available from ThermoFisher Scientific) (step 230).

In some embodiments, a directional concatemeric array is formed and used in the transfection of a packing cell line. In some embodiments, the formation of the directional array is achieved by utilizing the one or more restriction enzyme sites within the lentiviral transfer vector plasmid which flank the lentiviral vector backbone. In some embodiments, restriction digestion utilizes the restriction enzyme sites flanking the TL20c vector cassette and allows for the formation of nucleotide nonpalindromic overhangs, which can only be used to ligate from heat to tail. In some embodiments, directional ligation, according to the methods described herein, allow for the generation of a concatemeric array which comprises predominantly head-to-tail DNA products.

In some embodiments, the concatemeric array is formed according to the method set forth in Example 3 herein. Of course, the skilled artisan will recognize that the procedure provided in Example 3 may be adapted for the formation of a concatemeric array having different ratios of a first plasmid to a second plasmid and for transfer plasmids other than LVsh5/C46, e.g. transfer plasmids designed to express a gamma-globin gene or any other gene of interest.

In some embodiments, the concatemeric array is purified by phenol-extraction and ethanol precipitation prior to transfection into a packing cell line. While this conventional technique is inexpensive and effective, the procedure is, however, time consuming and may not yield reproducible yields. Itis also believed that there may be a risk of phenol/chloroform carry-over into the final sample when using this particular method. Moreover, the process is believed to involve further hazardous chemicals and may generate toxic waste that must be disposed of with care and in accordance with hazardous waste guidelines.

Alternatively, in other embodiments, a silica-based method is used to purify the newly synthesized concatemeric array after ligation. This method is believed to provide a simple, reliable, fast, and convenient way for isolation of the high-quality transfection-grade concatemeric array. In some embodiments, the concatemeric array is purified using a DNeasy Mini spin column, available from Qiagen, such as using the procedure set forth in Example 6.

Transfection/Single Clone Isolation

Following purification of the concatemeric array, the array is then used to transfect packaging cell line cells. As used herein, the terms “transformation” and “transfection” are intended to refer to a variety of art-recognized techniques for introducing foreign nucleic acid (e.g., DNA or RNA) into cells. As will be evident from the examples provided herein, when a host cell permissive for production of lentiviral particles is transfected with the generated concatemeric array, the cell becomes a producer cell, i.e. a cell that produces infectious lentiviral particles.

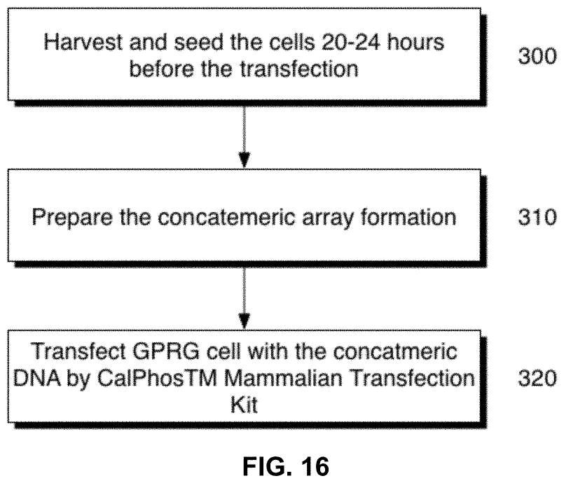

In general, the formed concatemeric array or formed directional concatemeric array may be introduced into cells via conventional transfection techniques. For example, and with reference to FIG. 16, in some embodiments, cells are harvested and seeded about 20 to about 24 hours before transfection (step 300) and then transfected (step 320) with the synthesized concatemeric array (step 310). A procedure for transfecting a packing cell line cell is provided in Example 4 herein.

One packaging cell line suitable for transfection with the formed concatemeric or directional concatemeric arrays is the GPR packaging cell line. The GPR line is an HIV-1-based packaging cell line derived from 293T/17 cells with the necessary viral components including gagpol and rev (see, Throm et al., Efficient construction of producer cell lines for a SIN lentiviral vector for SCID-X1 gene therapy by concatemeric array transfection. Blood 113: 5104-5110, the disclosure of which is hereby incorporated by reference herein in its entirety).

Another packaging cell line suitable for transfection with the formed concatemeric or directional concatemeric arrays is a GPRG packaging cell line. In some embodiments, the GPRG packing cell line comprises gagpol, rev, and VSV-G.

Yet another packaging cell line suitable for transfection with the formed concatemeric or directional concatemeric arrays is a GPRT packaging cell line (gagpol, rev, and tat). GPRG and GPRT packaging cell lines and methods of forming the same are also disclosed by Throm et. al, the disclosure of which is again hereby incorporated by reference herein in its entirety. Other suitable packaging cell lines (e.g. GPRT-G) are described by Wielgosz et al. “Generation of a lentiviral vector producer cell clone for human Wiskott-Aldrich syndrome gene therapy,” Molecular Therapy—Methods & Clinical Development 2, Article number: 14063 (2015), the disclosure of which is hereby incorporated by reference herein in its entirety.

The skilled artisan will appreciate that other packaging cell lines suitable for use with the presently disclosed method may also be utilized. In some embodiments, other packaging cell lines may be derived from any of the GPR, GPRG, GPRT, or GPRT-G packaging cell lines. Without wishing to be bound by any particular theory, it is believed that the GPRT-G cell line has higher transduction efficiency in CD34+ cells (see Wielgosz). As used here, the term “derived from,” refers to a population of cells clonally descended from an individual cell and having some select qualities, such as the ability to produce active protein at a given titer, or the ability to proliferate to a particular density.

In some embodiments, the packaging cell line cells are 293T cells. 293T cells (or HEK 293T) are human cell line cells, derived from the HEK 293 cell line, that expresses a mutant version of the SV40 large T antigen. Yet other suitable packaging cell line cells are described in U.S. Patent Publication No. 2009/0187997, in PCT Publication No. WO/2012/170431, and in U.S. Pat. No. 8,034,620, the disclosures of which are described herein by reference in their entireties. PCT Publication No. WO/2012/170431 describes packaging cells which may be prepared from CHO cells, BHK cells, MDCK cells, C3H 10T1/2 cells, FLY i, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells.

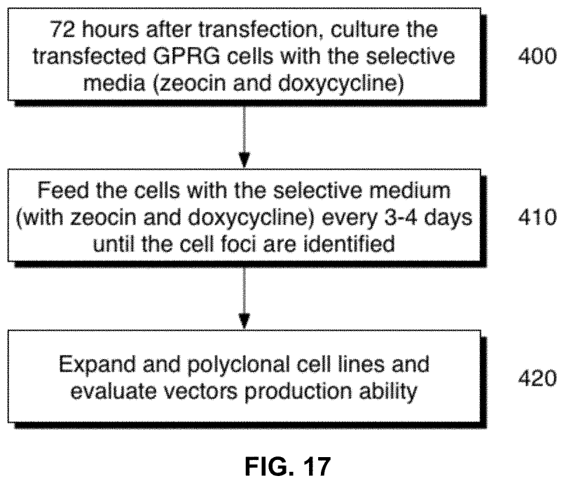

FIG. 17 illustrates the general process of selecting for transfected cells. In some embodiments, and after about 72 hours after transfection, GPRG cells are cultured with the selective media (zeocin and doxycycline) (step 400). The cells are then fed with selective medium (zeocin and doxycycline) every about 3 to about 4 days until the cell foci are identified (step 410). Subsequently, the cell lines are expanded and evaluated (step 420).

In some embodiments, and following transfection, a single foci selection/screening process is utilized to identify the single cell clones that have good manufacturing potential. According to this method, in some embodiments, selected cells are seeded sparsely in 150×25 mm dishes and allowed to expand and form discernible colonies for about 2 to about 3 weeks. The individual colonies may then be transferred to another smaller culture vessel for monoclonal expansion. This method is believed to be a cost-effective and frequently adopted technique; however, due to the nature limitations in the single foci selection technique, achieving a high probability of monoclonality of a good producing cell line may be challenging.

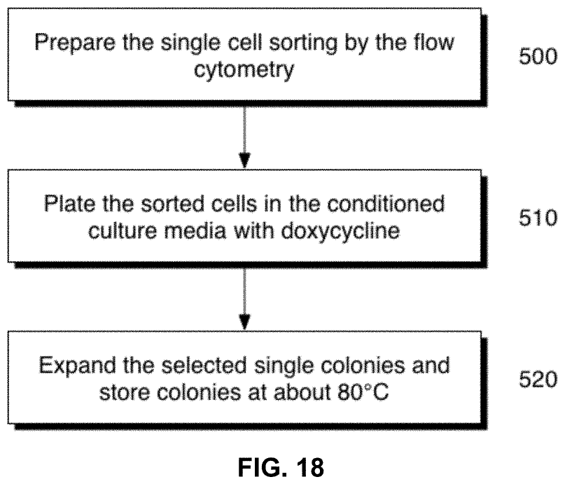

FIG. 18 illustrates single colony isolation. At step 500, flow cytometry is utilized to prepare the single cell sorting. The cells are then plated (step 510) in the conditioned culture media and expanded (step 520).

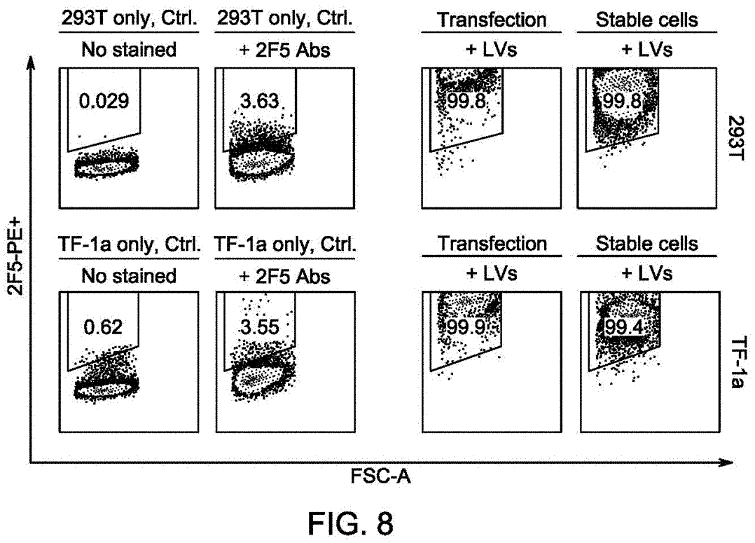

In other embodiments, in order to generate a high titer lentiviral vector stable producer cell line, a fluorescence activated cell sorter (FACS) is used to isolate single clones (see, e.g. FIG. 8). Conditioned medium, e.g. Zeocin (50 μg/mL) and Doxycycline (1 ng/mL), may also be added during a sorting process to increase cell attachment and viability, and promote colony formation. The use of conditioned growth media and the high throughput ability of FACS system is believed to enable the screening of a large number of clones and thus is believed to increases the probability of finding high titer lentiviral vector producer clones.

In some embodiments, a clone with good growing rate and viral production ability is tested for stability over about 20 passages.

Induction of Producer Cell Lines to Generate Viral Vectors and “Two-Day Harvesting”

Following the selection and expansion of the selected clones, the selected clones are induced to produce viral vectors, e.g. retroviral vectors, lentiviral vectors. In some embodiments, induction may be carried out according to the procedures known to those of ordinary skill in the art.

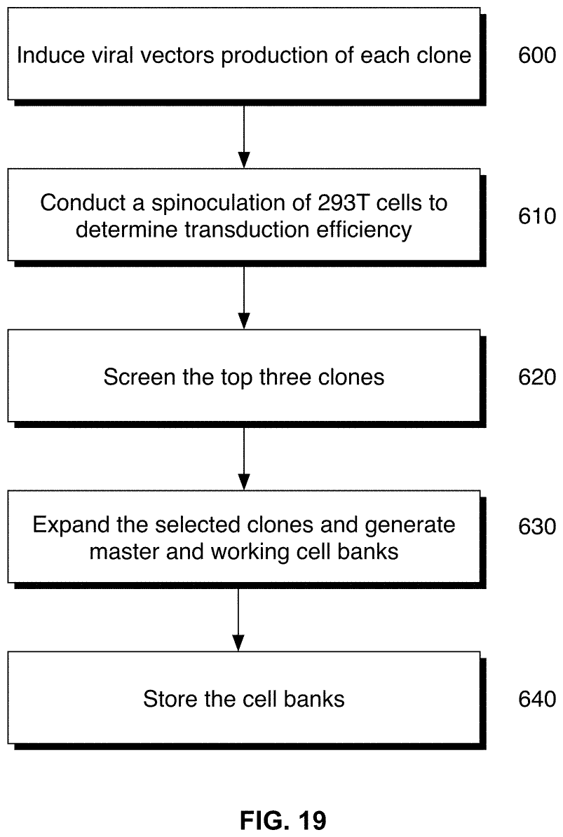

FIG. 19 illustrates the process of induction and evaluation. At step 600, the viral vectors are induced and then the spinoculation of cells, e.g. 293T cells, is conducted to determine transduction efficiency (step 610). In some embodiments, the top three clones are screened (step 620) and expanded (step 630). In some embodiments, the clones are then stored (e.g. under liquid nitrogen) (step 640). In some embodiments, the stored cell banks may then be utilized in the repeated harvesting of viral supernatant as described herein.

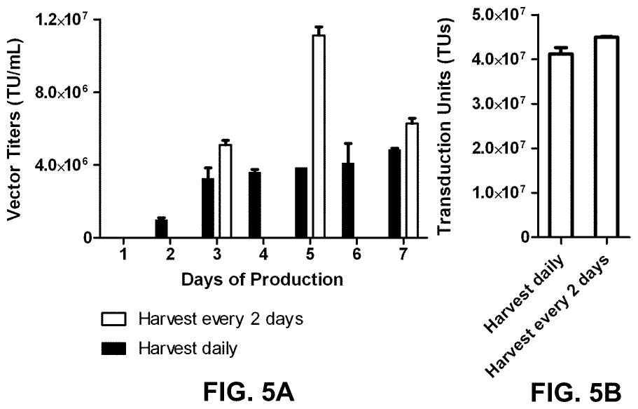

While a production protocol utilizing a daily harvest (e.g. harvesting every about 24 hours) may be utilized to produce a variety of test vectors at small scale, daily harvesting and media exchange is not economical when biomanufacturing is conducted at large-scale. This cost is amplified when using serum-containing media, which is more costly than serum-free media. As an alternative to a daily harvesting, a “two-day harvest” protocol has been devised as described herein. Applicant has unexpectedly discovered that a “two-day harvest” allows for the generation of about the same quantity of viral vectors as with the more traditional daily harvest, while also providing the benefit of requiring less culture medium. A comparison of vector titer yield from daily harvesting and according to a “two-day harvesting” protocol is illustrated in FIGS. 5A and 5B. In some embodiments, a “two-day harvest” procedure utilizes at least about 30% less culture medium as compared with traditional daily harvesting methods. In other embodiments, a “two-day harvest” procedure utilizes at least about 35% less culture medium as compared with traditional daily harvesting methods. In other embodiments, a “two-day harvest” procedure utilizes at least about 40% less culture medium as compared with traditional daily harvesting methods. In other embodiments, a “two-day harvest” procedure utilizes at least about 45% less culture medium as compared with traditional daily harvesting methods. In yet other embodiments, a “two-day harvest” procedure utilizes at least about 50% less culture medium as compared with traditional daily harvesting methods. In yet other embodiments, a “two-day harvest” procedure utilizes at least about 55% less culture medium as compared with traditional daily harvesting methods.

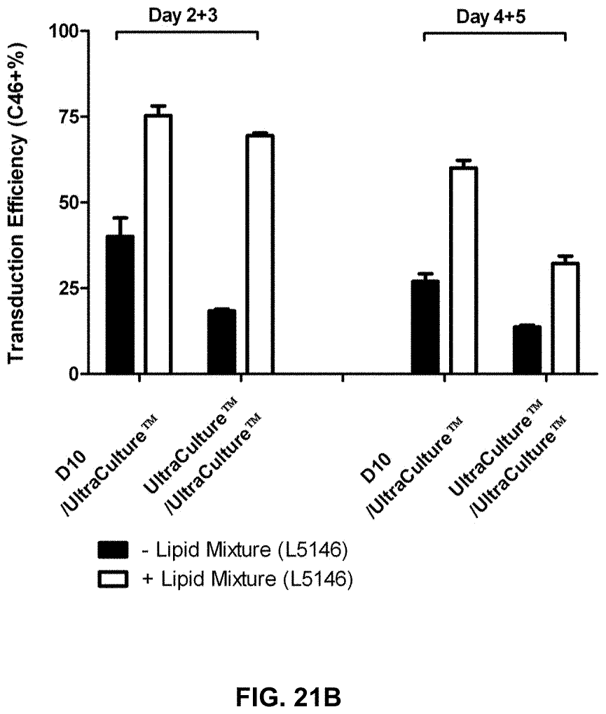

Applicant has further demonstrated that although viral vector titer may be reduced when repeatedly harvesting in a serum-free medium (in either or both the culture and/or production phases) as compared with the use of serum-containing media, that the reduction in viral vector titer is believed not to be significant (see FIGS. 21A and 21B), especially when considering that the use of a serum-free medium mitigates or prevents the risk of contamination from pathogenic agents possibly present in serum-containing media. In addition, Applicant has demonstrated that a “two-day harvest” protocol mitigates the reduction in viral titer when a switch from serum-containing media to serum-free media is made. In fact, Applicant has discovered that cells better tolerate a “two-day harvest” in serum-free media as compared with daily harvesting in serum-free media. Finally, the costs associated with performing biomanufacturing operations using a serum-free medium are comparatively far less than when performing biomanufacturing operations using a serum-containing medium, and thus any loss in viral vector titer when switching to a serum-free medium is manageable given the recognized cost savings (compare Tables A and B, herein).

As noted above, the processes of the present disclosure utilize a “two-day harvest” protocol where viral vector is repeated harvested about every two days following an initial harvesting of viral vectors. In some embodiments, the initial harvesting of the viral vectors occurs between about 24 hours to about 56 hours after induction (i.e. after inducing viral vector production). In some embodiments, the initial harvesting of the viral vectors occurs between about 30 hours to about 56 hours after induction (i.e. after inducing viral vector production). In some embodiments, the initial harvesting of the viral vectors occurs between about 40 hours to about 56 hours after induction (i.e. after inducing viral vector production). In other embodiments, the initial harvesting of the viral vectors occurs between about 42 hours to about 54 hours after induction. In yet other embodiments, the initial harvesting of the viral vectors occurs between about 44 hours to about 52 hours after induction. In further embodiments, the initial harvesting of the viral vectors occurs between about 46 hours to about 50 hours after induction. In further embodiments, the initial harvesting of the viral vectors occurs between about 47 hours to about 49 hours after induction. In yet further embodiments, the initial harvesting of the viral vectors occurs about 48 hours after induction. In some embodiments, the initial harvesting occurs at least 30 hours after induction. In some embodiments, the initial harvesting occurs at least 35 hours after induction. In some embodiments, the initial harvesting occurs at least 40 hours after induction. In some embodiments, the initial harvesting occurs at least 45 hours after induction.

In some embodiments, repeated harvesting according to the presently disclosed “two day harvest” protocol comprises repeatedly harvesting viral vectors every about 40 hours to about 56 hours following an initial harvesting of viral vector. In other embodiments, an initial harvest is conducted between about 40 and about 56 hours after induction of the stable producer cell line cells, and then harvesting is repeated every about 42 to about 55 hours thereafter. In yet other embodiments, an initial harvest is conducted between about 40 and about 56 hours after induction of the stable producer cell line cells, and then harvesting is repeated every about 44 to about 52 hours thereafter. In further embodiments, an initial harvest is conducted between about 40 and about 56 hours after induction of the stable producer cell line cells, and then harvesting is repeated every about 46 to about 50 hours thereafter. In even further embodiments, an initial harvest is conducted between about 40 and about 56 hours after induction of the stable producer cell line cells, and then harvesting is repeated about every 48 hours thereafter. Applicant has discovered that viral vectors could be harvested about 48 hours following induction and that the greatest quantity of viral titer could be yielded at about 72 hours after induction. Applicant has also discovered that the repeated virus harvesting protocol can also increase the final yield of viral vectors.

In some embodiments, an initial harvest is conducted between about 40 and about 56 hours after induction of the stable producer cell line cells, and then harvesting is repeated at least about every 40 hours thereafter. In some embodiments, an initial harvest is conducted between about 40 and about 56 hours after induction of the stable producer cell line cells, and then harvesting is repeated at least about every 42 hours thereafter. In some embodiments, an initial harvest is conducted between about 44 and about 56 hours after induction of the stable producer cell line cells, and then harvesting is repeated at least about every 40 hours thereafter. In some embodiments, an initial harvest is conducted between about 46 and about 56 hours after induction of the stable producer cell line cells, and then harvesting is repeated at least about every 40 hours thereafter.

In some embodiments, the serum-free media used for harvesting is replaced after each repeated harvesting. In some embodiments, no additional serum-free media is introduced to the generated stable producer cell line cells during each individual harvesting. In some embodiments, the repeated harvesting comprises adding fresh media to stable producer cell line cells without introducing additional stable producer cell line cells.

In some embodiments, the processes according to the present disclosure (“two day harvest” in serum-free media) allow for a viable viral titer to be harvested ranging from about 0.5×10⁶ TU/mL to about 5×10⁶ TU/mL during each individual harvesting of the repeated harvesting. In other embodiments, the processes according to the present disclosure (“two day harvest in serum-free media) allow for a viable viral titer to be harvested ranging from about 0.5×10⁶ TU/mL to about 4×10⁶ TU/mL during each individual harvesting of the repeated harvesting. In yet other embodiments, the processes according to the present disclosure (“two day harvest” in serum-free media) allow for a viable viral titer to be harvested ranging from about 0.5×10⁶ TU/mL to about 3.5×10⁶ TU/mL during each individual harvesting of the repeated harvesting. In further embodiments, the processes according to the present disclosure (“two day harvest” in serum-free media) allow for a viable viral titer to be harvested ranging from about 0.5×10⁶ TU/mL to about 3×10⁶ TU/mL during each individual harvesting of the repeated harvesting. In even further embodiments, the processes according to the present disclosure (“two day harvest” in serum-free media) allow for a viable viral titer to be harvested ranging from about 0.5×10⁶ TU/mL to about 2.5×10⁶ TU/mL during each individual harvesting of the repeated harvesting. In yet other embodiments, the processes according to the present disclosure (“two day harvest” in serum-free media) allow for a viable viral titer to be harvested ranging from about 0.5×10⁶ TU/mL to about 2×10⁶ TU/mL during each individual harvesting of the repeated harvesting. In yet other embodiments, the processes according to the present disclosure (“two day harvest” in serum-free media) allow for a viable viral titer to be harvested ranging from about 0.5×10⁶ TU/mL to about 1.7×10⁶ TU/mL during each individual harvesting of the repeated harvesting. In yet further embodiments, the processes according to the present disclosure (“two day harvest” in serum-free media) allow for a viable viral titer to be harvested ranging from about 0.5×10⁶ TU/mL to about 1.6×10⁶ TU/mL during each individual harvesting of the repeated harvesting. In yet other embodiments, the processes according to the present disclosure “(two day harvest” in serum-free media) allow for a viable viral titer to be harvested ranging from about 0.5×10⁶ TU/mL to about 1.4×10⁶ TU/mL during each individual harvesting of the repeated harvesting. In yet other embodiments, the processes according to the present disclosure (“two day harvest” in serum-free media) allow for a viable viral titer to be harvested ranging from about 0.5×10⁶ TU/mL to about 1.3×10⁶ TU/mL during each individual harvesting of the repeated harvesting. In still further embodiments, the processes according to the present disclosure (“two day harvest” in serum-free media) allow for a viable viral titer to be harvested ranging from about 0.5×10⁶ TU/mL to about 1.2×10⁶ TU/mL during each individual harvesting of the repeated harvesting. In even further embodiments, the processes according to the present disclosure “(two day harvest” in serum-free media) allow for a viable viral titer to be harvested ranging from about 0.5×10⁶ TU/mL to about 1.1×10⁶ TU/mL during each individual harvesting of the repeated harvesting. In even further embodiments, the processes according to the present disclosure “(two day harvest” in serum-free media) allow for a viable viral titer to be harvested ranging from about 0.5×10⁶ TU/mL to about 1×10⁶ TU/mL during each individual harvesting of the repeated harvesting.

In some embodiments, the processes according to the present disclosure (“two day harvest” in serum-free media) allow for a viable viral titer of at least about 0.5×10⁶ TU/mL to be harvested during each individual harvesting of the repeated harvesting. In some embodiments, the processes according to the present disclosure (“two day harvest” in serum-free media) allow for a viable viral titer of at least about 1×10⁶ TU/mL to be harvested during each individual harvesting of the repeated harvesting. In some embodiments, the processes according to the present disclosure (“two day harvest” in serum-free media) allow for a viable viral titer of at least about 1.5×10⁶ TU/mL to be harvested during each individual harvesting of the repeated harvesting. In some embodiments, the processes according to the present disclosure (“two day harvest” in serum-free media) allow for a viable viral titer of at least about 2×10⁶ TU/mL to be harvested during each individual harvesting of the repeated harvesting. In some embodiments, the processes according to the present disclosure (“two day harvest” in serum-free media) allow for a viable viral titer of at least about 2.5×10⁶ TU/mL to be harvested during each individual harvesting of the repeated harvesting. In some embodiments, the processes according to the present disclosure (“two day harvest” in serum-free media) allow for a viable viral titer of at least about 3×10⁶ TU/mL to be harvested during each individual harvesting of the repeated harvesting. In some embodiments, the processes according to the present disclosure (“two day harvest” in serum-free media) allow for a viable viral titer of at least about 3.5×10⁶ TU/mL to be harvested during each individual harvesting of the repeated harvesting. In some embodiments, the processes according to the present disclosure (“two day harvest” in serum-free media) allow for a viable viral titer of at least about 4×10⁶ TU/mL to be harvested during each individual harvesting of the repeated harvesting. In some embodiments, the processes according to the present disclosure (“two day harvest” in serum-free media) allow for a viable viral titer of at least about 4.5×10⁶ TU/mL to be harvested during each individual harvesting of the repeated harvesting. In some embodiments, the processes according to the present disclosure (“two day harvest” in serum-free media) allow for a viable viral titer of at least about 5×10⁶ TU/mL to be harvested during each individual harvesting of the repeated harvesting.

In some embodiments, a production phase lasts for between about 5 days to about 90 days. In some embodiments, a production phase lasts for between about 5 days to about 80 days. In some embodiments, a production phase lasts for about 5 days to about 70 days. In some embodiments, a production phase lasts for between about 5 days to about 60 days. In some embodiments, a production phase lasts for between about 5 days to about 50 days. In some embodiments, a production phase lasts for about 5 days to about 40 days. In some embodiments, a production phase lasts for between about 5 days to about 30 days. In some embodiments, a production phase lasts for between about 5 days to about 20 days. In some embodiments, a production phase lasts for between about 10 days to about 90 days. In some embodiments, a production phase lasts for between about 10 days to about 60 days. In some embodiments, a production phase lasts for between about 10 days to about 45 days. In some embodiments, the production phase lasts for at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, or at least about 90 days. In some embodiments, the production phase lasts for about 5, about 10, about 15, about 20, about 25, about 30, about 35, about 40, about 45, about 50, about 55, about 60, about 65, about 70, about 75, about 80, about 85, or about 90 days.

In some embodiments, harvesting occurs at least twice. In other embodiments, harvesting occurs at least three times. In other embodiments, harvesting occurs at least four times. In other embodiments, the repeated harvesting occurs at least five times. In other embodiments, harvesting occurs at least six times. In other embodiments, harvesting occurs at least seven times. In other embodiments, harvesting occurs at least eight times. In other embodiments, harvesting occurs at least nine times. In other embodiments, harvesting occurs at least ten times. In some embodiments, harvesting occurs at eleven twice. In other embodiments, harvesting occurs at least twelve times. In other embodiments, harvesting occurs at least thirteen times. In other embodiments, harvesting occurs at least fourteen times. In other embodiments, harvesting occurs at least fifteen times. In other embodiments, harvesting occurs at least sixteen times. In other embodiments, harvesting occurs at least seventeen times. In other embodiments, harvesting occurs at least eighteen times. In other embodiments, harvesting occurs at least nineteen times. In other embodiments, harvesting occurs at least twenty times. In other embodiments, harvesting occurs at least twenty-five times.

Applicant has also discovered that repeatedly harvesting viral vector in a serum-free medium every about two days allows for substantially the same quantity of viral vector titer to be harvested as compared with the quantity of viral vector titer recovered when using the same harvesting protocol but in serum-containing media (see, e.g., Tables A and B; see also FIGS. 21A and 21B). In some embodiments, the quantity of infectious particles produced in a serum-free medium (following a “two-day harvest” schedule) is within at least about 65% of the total quantity of infectious particles produced in a serum-containing medium (following the “two-day harvest” schedule). In some embodiments, the quantity of infectious particles produced in a serum-free medium (using a “two-day harvest” schedule) is within at least about 70% of the total quantity of infectious particles produced in a serum-containing medium (using a “two-day harvest” schedule). In some embodiments, the quantity of infectious particles produced in a serum-free medium (using a “two-day harvest” schedule) is within at least about 75% of the total quantity of infectious particles produced in a serum-containing medium (using a “two-day harvest” schedule). In some embodiments, the quantity of infectious particles produced in a serum-free medium (using a “two-day harvest” schedule) is within at least about 80% of the total quantity of infectious particles produced in a serum-containing medium (using a “two-day harvest” schedule). In some embodiments, the quantity of infectious particles produced in a serum-free medium (using a “two-day harvest” schedule) is within at least about 85% of the total quantity of infectious particles produced in a serum-containing medium (using a “two-day harvest” schedule). In some embodiments, the quantity of infectious particles produced in a serum-free medium (using a “two-day harvest” schedule) is within at least about 90% of the total quantity of infectious particles produced in a serum-containing medium (using a “two-day harvest” schedule). In some embodiments, the quantity of infectious particles produced in a serum-free medium (using a “two-day harvest” schedule) is within at least about 95% of the total quantity of infectious particles produced in a serum-containing medium (using a “two-day harvest” schedule).

In some embodiments, and following the conclusion of each repeated harvesting step, the harvested viral vectors may be purified through filtration. In some embodiments, the harvested vectors are characterized by determining one or more of viral titer, viral copy per cell genome, and/or p24 concentration.

Examples of Repeated Harvesting Using Serum-Containing Media

First Exemplary Method

In a first exemplary method, a medium containing serum is utilized at each step, i.e. in a culture phase, in production phase, and during passaging of the cells. In some embodiments, the medium containing serum is a D10 serum-containing medium. In some embodiments, a D10 serum-containing medium comprises Dulbecco's modified Eagle's medium (DMEM) and fetal bovine serum, heat-inactivated (10%) (i.e. a fetal bovine serum that is already heat inactivated prior to use).

In one embodiment is a first method of generating viral vectors using a two-day harvest according to the present disclosure, the first method comprising the following steps:

(1) Remove the old culture medium of the producer lines culture dish as completely as possible and wash the cells with 1×PBS.

(2) Add TrypLE™ Express Enzyme (1×) to the culture dish (available from ThermoFisher Scientific).

(3) Place in an incubator at about 37° C. for about 2 minutes.

(4) Wash out the cells by adding D10 medium (free from any drug) and dissociate the cell clusters into single cells by pipetting up and down (D10 media: Dulbecco's Modified Eagle Medium with high glucose, GlutaMAX™ Supplement and 10% (w/v) FBS and 1% (w/v) Pen/Strep).

(5) Centrifuge the cells for about 5 minutes at about 4° C. at about 1200 rpm (or 125×g).

(6) Aspirate the medium and gently suspend the formed pellet in fresh D10 medium (no drug).

(7) Seed the cells at about 95% confluent in a culture dish (Roughly by plating 4×10⁶ Cells/60-mm culture dish, Vectors Induction).

(8) The seeded cells may then be supplemented with fresh, pre-warm D10 medium after about 24 hr (Day 1 post-induction).

(9) Harvest the viral vectors for the first time about 48 hours after the first-time media change (Day 3 post-induction).

(10) Add the fresh, pre-warm medium to the culture dish.

(11) Conduct the second time viral vector harvested about 48 hours after the second time medium change (Day 5 post-induction).

(12) Add the fresh, pre-warm medium to the culture dish.

(13) Conduct the third time viral vector harvested about 48 hours after the third time medium change (Day 7 post-induction).

Second Exemplary Method

In a second exemplary method, serum-containing media is utilized at each step, i.e. during passaging of the cells, in a culture phase, and in a production phase. In some embodiments, the serum-containing medium is D10.

(1) Remove the media of the producer lines culture dish as completely as possible and wash the cells with 1×PBS.

(2) Gently Pipette 1×TrypLE Express onto the washed cell monolayer using 3 mL for 100 mm culture dish.

(3) Rotate flask to cover the monolayer with TrypLE Express.

(4) Return flask to the incubator and leave for about 2 minutes.

(5) Gently tapped side of the flasks to release any remaining attached cells.

(6) Re-suspend the cells in about 2 mL of the fresh D10 media (no antibiotics) and transfer to a 15 mL conical centrifuge Tube.

(7) Centrifuge the cells for about 5 minutes at about 1200 rpm (or 125×g).

(8) Aspirate media and gently suspend pellet in about 5 mL fresh D10 culture media (no antibiotics).

(9) Determine the cell counts by TC10™ Automated Cell Counter.

(10) Seed the cells about greater than about 95% confluent at culture dish (by plating about 4×10⁶ Live Cells in 60-mm culture dish).

(11) The seeded cells were supplemented with fresh, warmed-up D10 media daily (every about 24 hours).

Applicant has discovered that viral vectors could be harvested from the cells about 48 hours post induction and that the highest viral titer could be yielded at 72 hours after induction. Applicant has again unexpectedly discovered that viral vectors could be harvested from days about 2 to about 4 days post induction.

In some embodiments, the harvested vectors are purified through filtration. In some embodiments, the harvested vectors are characterized by determining viral titer, viral copy per cell genome, and p24 concentration.

Examples of Repeated Harvesting Using Serum-Free Media

The present disclosure also provides methods of conducting a two-day harvest using serum-free media. As detailed in the third and fourth embodiments described below, serum-free media may be utilized in at least one of a culture phase or a production phase.

Applicant has discovered a cost-effective method of repeatedly harvesting viral vector with only a minimal reduction in titer, by combining a “two-day harvest” procedure with the use of serum-free media. It is believed that the use of serum-free media together with a “two-day harvest” protocol is especially important for large-scale bio-manufacturing where costs of serum-containing media may be quite high. Accordingly, in some embodiments, harvesting is conducted at least partially in a serum-free medium. For example, at least one of a culture phase or a production phase utilizes serum-free media. By way of another example, both the culture phase and the production phase utilize serum-free media.

TABLE A
Compares the use of serum-free media and serum-containing media when
harvesting viral titer on a daily basis. A change in viral titer is observed
when the media used for the culture and/or production phases are changed.

| Media Utilized | Viral Titer | % Reduction |
|---|---|---|
| (Culture/Production) | (TU/mL) | in Titer |
| Serum-containing D10 | 5.09E+06 | NA |
| medium/Serum-containing | | |
| D10 medium | | |
| Serum-containing D10 | 1.47E+06 | 70% |
| medium/Serum-free | | |
| UltraCULTURE medium with | | |
| added lipids | | |
| Serum-free UltraCULTURE | 1.32E+06 | 74% |
| medium/Serum-free | | |
| UltraCULTURE medium with | | |
| added lipids | | |

*Lipid Mixture cost $0.61 per Liter of VCM

TABLE B
Compares the use of serum-free media and serum-containing media
during the production phase when harvesting viral titer about every
48 hours (“two-day harvest” process). The stable producer cell line
cells were induced with serum-containing D10 media by removing
doxycycline. One day after induction, the media was changed to one
of pre-warmed (i) serum-containing D10 media; (ii) serum-free
media; or (iii) serum-free media including EX-CYTE. Titer was
harvested every two days for each batch (batch 1-days 2 and 3;
batch 2-days 4 and 5; batch 3-days 6 and 7). Viral titer was
analyzed 5-days after transduction.

| | | | | Total | |
|---|---|---|---|---|---|
| | | | | Infections | |
| | Titer (10⁶ TU/mL) | | | particles | Re- |
| Production | Batch | Batch | Batch | (10⁶ TU/ | duc- |
| Condition | 1 | 2 | 3 | mL) | tion |
| D10 | 33.5 ± 4.6 | 28.9 ± 6.0 | 4.68 ± 4.13 | 268.4 ± 22.0 | NA |
| Serum- | | | | | |
| containing | | | | | |
| Medium | | | | | |
| Ultra- | 28.5 ± 4.3 | 19.7 ± 2.2 | 3.08 ± 0.11 | 204.8 ± 26.5 | 24% |
| CULTURE ™ | | | | | |
| Serum-free | | | | | |
| Medium | | | | | |
| Ultra- | 37.0 ± 8.2 | 15.3 ± 3.5 | 7.78 ± 3.33 | 240.2 ± 60.0 | 10% |
| CULTURE ™ | | | | | |
| Serum-free | | | | | |
| Medium + | | | | | |
| EXCYTE | | | | | |

D10 Medium: DMEM/GlutaMAX ™ + 10% FBS P/S;
Batch 1: Days 2 and 3;
Batch 2: Days 4 and 5;
Batch 3: Days 6 and 7

Third Exemplary Method

In a third exemplary method, serum-free media is used in some steps (e.g. in both culture and production phases) while serum-containing media is used in other steps (e.g. during passaging of the cells). In some embodiments, the serum-containing media is D10 serum-containing media; and the serum-free media is UltraCULTURE™ media (available from Lonza). In some embodiments, the serum-free media may optionally include one or more growth factors and/or lipids, e.g. “Lipid Mixture Supplement,” available from Sigma L5145). Both the UltraCULTURE™ medium and the Sigma L5145 medium stabilized cells during vector production. Comparative data is provided in Tables A and B (above) and in FIGS. 21A and 21B.

Accordingly, the present disclosure provides a third method of generating viral vectors using a two-day harvest, the third method comprising the following steps:

(1) Passage the cells at least 4 times after thawing (e.g. to recover from freezing) before using them in the viral vector production.

(2) Culture the cells to the desired quantity (passage the cells every other day)

(3) Remove the medium of the producer lines culture dish as completely as possible and wash the cells with 1×PBS.

(4) Gently Pipette 1×TrypLE Express onto the washed cell monolayer

(5) Rotate flask to cover the monolayer with TrypLE Express.

(6) Return flask to the incubator and leave for about 2 minutes.

(7) Gently tap the side of the flasks to release any remaining attached cells.

(8) Resuspend the cells in the fresh D10 medium (no antibiotics) and transfer to a conical centrifuge Tube.

(9) Centrifuge cells for about 5 minutes at about 1200 rpm.

(10) Aspirate the medium and gently suspend the pellet in the fresh D10 culture medium (no antibiotics).

(11) Determine the cell counts using a TC10™ Automated Cell Counter.

(12) Seed the cells>95% confluent at culture dish directly into UltraCULTURE™ serum free medium (no antibiotics).

(13) At about 24 hours after induction, the old culture medium is replaced with fresh, pre-warm UltraCULTURE™ serum-free medium.

(14) Viral vectors were harvested from the induced cells about 72-hour post induction.

Fourth Exemplary Method

In a fourth exemplary method, a serum-free medium is used in the production phase; while a serum-containing medium is used in both the culture phase and during passaging of the cells. In some embodiments, the medium containing serum is a D10 serum-containing medium; and the serum-free medium is a UltraCULTURE™ medium (available from Lonza) (optionally comprising addition growth factors, e.g. EX-CYTE®). The EX-CYTE® supplement is a water-soluble concentrate of cholesterol, lipoproteins, and fatty acids that provides a balanced profile of metabolic factors proven to enhance cell growth and protein production in a variety of mammalian cells. In some embodiments, about 1% of v/v of EX-CYTE was added to the culture medium. It is believed that when using serum-free media in the production phase, cells did not need to proceed through an adaption procedure. On the other hand, it is believed that cells needed some time to adapt to the use of serum-free media when such media was used in a culture phase.

In another embodiment is a fourth method of generating viral vectors using a two-day harvest procedure according to the present disclosure, the fourth method comprising the following steps:

(1) Passage the cells at least about 4 times after thawing before using them in the viral vector production.

(2) Passage the cells daily at least two days before vector induction (To maintain log phase growth).

(3) Remove the medium of the producer lines culture dish as completely as possible and wash the cells with 1×PBS.

(4) Gently Pipette 1×TrypLE Express onto the washed cell monolayer.

(5) Rotate flask to cover the monolayer with TrypLE Express.

(6) Return flask to the incubator and leave for about 2 minutes.

(7) Gently tapped side of the flasks to release any remaining attached cells.

(8) Resuspend the cells in the fresh D10 medium (no antibiotics) and transfer to a conical centrifuge tube.

(9) Centrifuge cells for about 5 minutes at about 1200 rpm (or 125×g.

(10) Aspirate medium and gently suspend pellet in the fresh D10 culture medium (no antibiotics).

(11) Determine the cell counts by TC10™ Automated Cell Counter.

(12) Seed the cells>95% confluent at culture dish in the fresh D10 culture medium (no antibiotics).

(13) At about 24 hours after induction, the D10 culture medium is replaced with fresh, pre-warm UltraCULTURE™ serum free medium.

(14) Viral vectors were harvested from the induced cells at about 72, about 120, and about 168-hour post induction.

### EXAMPLES

### Example 1—Detailed Comparison of Self-Inactivating Lentiviral Vectors Produced by Transient Transfection and Vectors Produced by the Disclosed Stable Cell Line Method

The methods described herein were used to generate a stable cell line for the production of LVsh5/C46, a self-inactivating lentiviral vector (SIN-LV) encoding a short hairpin RNA (shRNA) for down-regulation of the HIV-1 co-receptor CCR5, in combination with the HIV-1 fusion inhibitor, C46. This lentiviral vector, produced by transient transfection, is currently being evaluated in clinical trials in HIV-infected individuals. Here, a comparative analysis of LVsh5/C46 produced by transient transfection and LVsh5/C46 produced using the methods described herein to support the application of this system for clinical manufacturing of LVsh5/C46 and other SIN-LVs was conducted.

The skilled artisan will appreciate that the methods described herein may be extended to the production of other self-inactivating lentiviral vectors. For example, the SIN-LV may include (i) a first nucleic acid sequence encoding an RNAi, an antisense oligonucleotide, or an exon skipping agent targeting an HPRT gene; and (ii) a second nucleic acid sequence encoding a therapeutic gene. In some embodiments, the second nucleic acid encoding the therapeutic gene is one which may genetically correct sickle cell disease or β-thalassemia; or reduce symptoms thereof (including the symptoms of severe sickle cell disease). In other embodiments, the nucleic acid encoding the therapeutic gene is one which may genetically correct immune deficiencies, hereditary diseases, blood diseases (e.g. hemophilia, hemoglobin disorders), neurological diseases, and/or lysosomal storage diseases; or reduce symptoms thereof. In some embodiments, the therapeutic gene is gamma globin gene. In some embodiments, the second nucleic acid sequence encoding the gamma globin gene is a hybrid gamma globin gene including a point mutation that confers a competitive advantage for the alpha-globin chain, skewing the formation of tetrameric HbF versus HbS.

Lentiviral vectors (LVs) were produced by calcium phosphate transfection in 293T cells using the 4-plasmid system (one transfer vector, two packaging vectors, and one envelope vector). Virus-containing media (VCM) was harvested 48 h post-transfection and concentrated by ultracentrifugation through a 20% sucrose cushion.

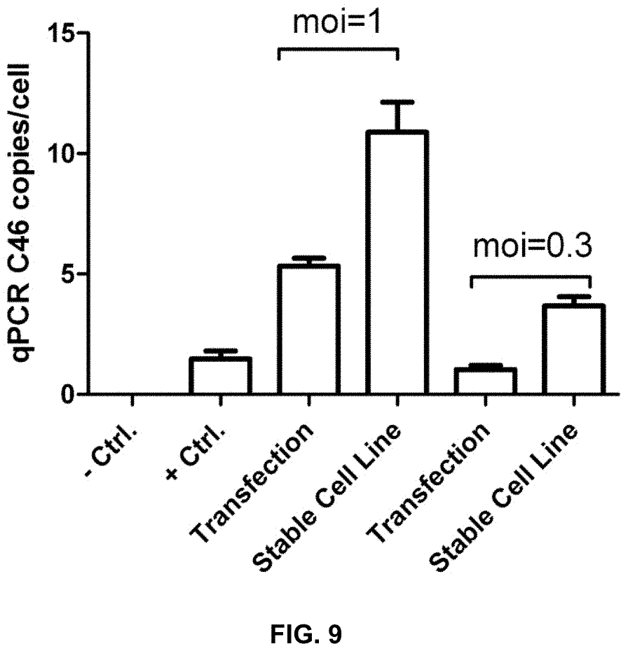

For cell line production, producer cell line cells were induced in media without doxycycline (Dox), and the VCM was harvested at about 72 h and similarly concentrated by ultracentrifugation. With reference to Table 1 and FIGS. 8 and 9, lentiviral vectors produced by each method were compared based on particle titer and using three independent assays for gene transduction potency on 293T and the TF-1a T cell line. These included FACS assays for cell surface C46 expression and shRNA-mediated knockdown of CCR5 expression, as well as a qPCR assay for vector copy number (VCN) per host cell genome. For all assays, titer was determined over a range of vector dilutions to define a linear relationship. The qPCR assay utilized genomic DNA extracted from transduced cells and detect the C46 transgene and a sequence from the endogenous β-globin gene. As such, C46 VCN could be normalized to cellular genome.

TABLE 1
Stable vs. Transient viral vector production.

| | | Titer 293T | Titer TF-1a | p24 |
|---|---|---|---|---|
| VCM² | Method | (TU/mL) | (TU/mL) | (ng/mL) |
| LVsh5/C46 | Transient Transfection | 2.78 × 10⁸ | 2.64 × 10⁸ | 5250 |
| TL20sh5/C46 | Stable Producer Cell | 1.40 × 10⁸ | 1.46 × 10⁸ | 13430 |

Abbreviation: TU, Transduction Unit;
VCM, virus-containing medium
VCM were concentrated 100-fold through a 20% sucrose cushion by ultracentrifugation

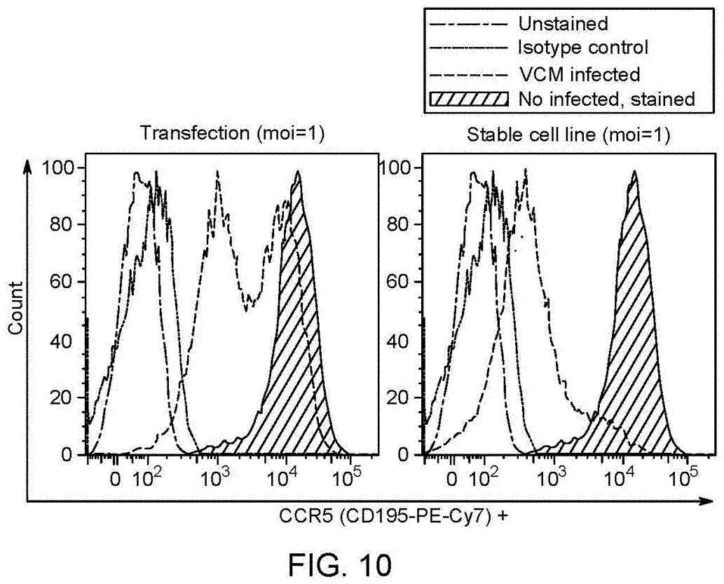

A higher concentration of p24 was observed in VCM produced by producer cell lines relative to transient transfection method. However, yield and potency of LVsh5/C46 produced using the two different systems was similar. Vectors were first evaluated for C46 titer by FACS using equal volumes of VCM. While vector produced by transient transfection had a modestly increased titer, when C46 titers were normalized and vector preparations were assessed for gene transduction using the qPCR assay or via functional knock-down of CCR5, vector produced by the stable producer cell lines showed greater potency (see Table 2). Down-regulation of CCR5 expression and genomic C46 transgene (VCN) were each significantly higher in the target cells treated with LVsh5/C46 produced by the methods disclosed herein than treated with vector produced by transient transfection (see Table 3 and FIG. 10).

TABLE 2
Analysis of C46 by qPCR in transduced cells.

| Condition | MOI³ | C46 copies/cells |
|---|---|---|
| no virus, negative ctrl. | — | ND |
| TF-1a², positive ctrl. | — | 1.36 ± 0.58 |
| Transient Transfection | 1 | 5.34 ± 0.55 |
| Stable Producer Line | 1 | 10.7 ± 2.17 |
| Transient Transfection | 0.3 | 1.01 ± 0.30 |
| Stable Producer Line | 0.3 | 3.67 ± 0.66 |

Abbreviation: ND, Not Detected;
MOI, Multiplicity of Infection
LVsh5/C46 single copy cell line
MOI based on C46 transduction titer

TABLE 3
Analysis of C46 by qPCR in transduced cells.

| Condition | MOI³ | C46 copies/cells |
|---|---|---|
| no virus, negative ctrl. | — | ND |
| TF-1a², positive ctrl. | — | 1.36 ± 0.58 |
| Transient Transfection | 1 | 5.34 ± 0.55 |
| Stable Producer Line | 1 | 10.7 ± 2.17 |
| Transient Transfection | 0.3 | 1.01 ± 0.30 |
| Stable Producer Line | 0.3 | 3.67 ± 0.66 |

Abbreviation: ND, Not Detected;
MOI, Multiplicity of Infection
LVsh5/C46 single copy cell line
MOI based on C46 transduction titer

Based on three independent assays, it has been demonstrated that the methods described herein provide a stable lentiviral vector production system which is capable of generating a similar quality and quantity of SIN-LVs compared to transient transfection methods. The higher CCR5 down-regulation efficacy and C46 VCN in transduced cells (normalized to C46 titer) indicated that LVsh5/C46 produced by producer cells had better potency than those vectors generated using the conventional 4-plasmid transient transfection method. By removing the tedious transient transfection step, without wishing to be bound by any particular theory, it is believed that this production system can be easily adapted to cG1VIP conditions for the manufacture of clinical grade materials for use in humans.

### Example 2—Development and Characterization of GPRG-Based Producer Cell Lines for the Bio-Production of Lentiviral Vectors for HIV Gene Therapy

The GPRG cell line system has previously been established for the clinical production of self-inactivating lentiviral vectors (SIN-LVs). Here, producer cell lines based on GPRG for the production of LVsh5/C46 were developed (LVsh5/C46 is a SIN-LV currently being assessed in the clinic for treatment of HIV-infected individuals). This vector encodes two viral entry inhibitors; sh5, a short hairpin RNA to the HIV co-receptor CCR5, and C46, a viral fusion inhibitor. Additionally, the stability of the GPRG packaging cell line, the GRPG-based LVsh5/C46 producer cell line, and LVsh5/C46 production following tetracycline induction as required for regulatory filling and clinical application of the GPRG system for bio-production of LVsh5/C46 was defined through this experimentation.

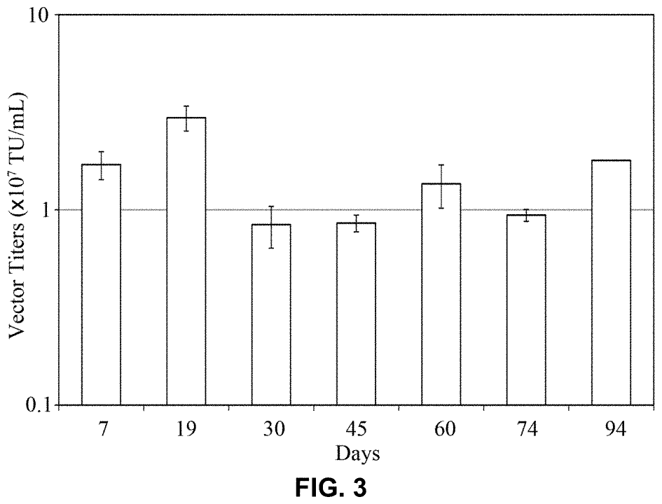

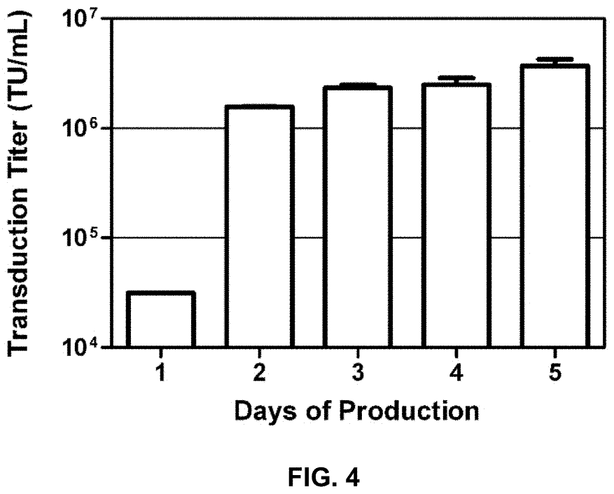

GPRG cells were cultured in D10 media with doxycycline (Dox) and puromycin (Puro). To generate LVsh5/C46 producer cells, GPRG cells were transfected with the transfer plasmid TL20-LVsh5/C46 and a Zeocin-resistance plasmid as a concatemeric array. Individual clones were evaluated for their ability to produce LVsh5/C46 vector and maintained in D10 media with Dox, Puro, and Zeocin. To assess the stability of the parental GPRG cell line for lentivirus (LV) production, GPRG cells were transfected with transfer vector every 10 passages over a 3-month period (50+ total passages) (see FIGS. 3A and 3B). Virus-containing media (VCM) was harvested 48 h post-transfection and vector titer was assessed by complementary gene transduction assays. To assess the stability of LV production from the stable producer cell clones, cells were induced in D10 media without Dox. VCM was harvested 72 h after induction and titer was similarly assessed over a range of vector dilutions. To analyze the stability of VSV-G expression following induction after long-term passage, GPRG cells were induced by Dox withdraw and then stained using a biotin-conjugated anti-VSV-G antibody, followed by a secondary staining with Streptavidin-Phycoerythrin.

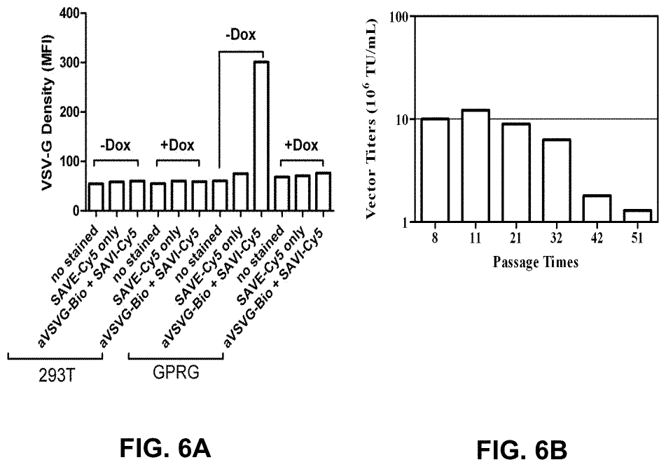

GPRG cells demonstrated stringent tetracycline-regulated expression of VSV-G. This packaging cell line was able to produce up to 10⁷ LV transduction units (TU)/mL after transfection with the LV transfer vector and maintained high-level LV production for more than 50 passages in continuous culture (see FIGS. 6A and 6B). By utilizing concatemeric array transfection, efficient construction of a producer cell line based on GPRG for the production of LVsh5C46 was demonstrated. This cell line consistently generated titers above 10⁶ TU/mL Further increases in titer could be achieved by re-cloning and selection of secondary producer cell lines. Titers peaked 2 to 5 days post-induction. It has been shown that the established stable producer cell lines maintained LVsh5/C46 production with titers exceeding 10⁶ TU/mL during continuous culture exceeding 25 passages.

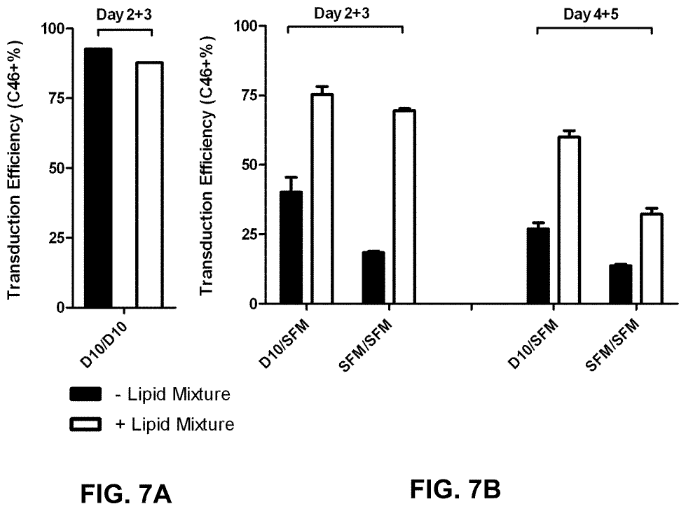

The GPRG cell line efficiently expressed VSV-G on cell surfaces upon the removal of Dox. It is also believed that these cell lines could generate high LVs titer after transfection of transfer vector plasmids. Moreover, this cell line allowed the derivation of high-titer producer cell lines for SIN-LVs. Producer cell lines demonstrated stable vector production during prolonged culture, and evaluation of the ability to adapt vector production to serum-free and suspension culture systems has been explored (see FIGS. 7A and 7B).

### Example 3—Protocol for the Generation of a Concatemeric Array

Step 1

Prepare 500 mL of 1×TAE running buffer by combining 490 mL of Deionized water with 10 mL 50×TAE ((Tris-acetate-EDTA) buffer).

Make the 1% agarose gel by adding 1 g of Agarose and 100 mL of 1×TAE buffer (Add 2 mL 50×TAE with 98 mL Autoclaved water) into a beaker and microwaving the mixture until there is no solid particles or bubbles (about 2.5 minutes).

Allow the mixture to cool for 3 minutes.

Add 10 μL of GelRed™ into the Agarose gel mixture and stir (available from Biotium).

Assemble the gel caster and gel comb. Pour the mixture into the gel mold and let it cool for 30 minutes (capacity: 60 μL for big comb)

Once the gel is cooled, fill the box with 1×TAE buffer until the gel is completely submerged.

Prepare the digest reaction mixture at room temperature to linearize the DNA

Digest 25 μg of the vector plasmid with the restriction enzyme SfiI. In a separate reaction, digest the resistance cassette plasmid PGK-ble with PflMI (10 μg is more than enough).

| Component | The ble marker | | Vector | |
|---|---|---|---|---|
| DNA Name | PGK-ble | | | |
| 10 FastDigest Green | 5 | μL | 10 | μL |
| Buffer | | | | |
| Plasmid DNA | 10 | μg | 25 | μg |
| FastDigest Enzyme 1: | 5 | μL | 0 | μL |
| PflMI | | | | |
| FastDigest Enzyme 2: | 0 | μL | 5 | μL |
| SfiI | | | | |
| Water, nuclease-free | To 50 | | To 100 | |
| Total Volume | 50 | μL | 100 | μL |

Mix gently and incubate at 37° C. in a heat for 15 min.

Add 10 μL of GeneRuler 1 kb plus DNA ladder mixture (2 μL DNA ladder+8 μL nuclease-free water) and 50 μL of sample mix into the available slots.

Turn on the electrophoresis machine and run with the voltage of 150 V for 1 hour.

Transfer the gel into the “UVP PhotoDoc-It” imaging system and obtain the image of the result.

Download the gel pictures from the Eye-Fi website.

Determine the DNA concentration of each sample using the NanoDrop 2000 Spectrophotometer.

Step 2

Cut DNA bands out of the Agarose gel.

Add 3 volumes of Buffer QG to 1 volume of gel (Generally add 500 μL QG).

Incubate at 50° C. for 10 minutes after the gel slice has dissolved completely.

Apply the sample to the QIAquick column, and centrifuge for 1 min at 17,900 rpm (available from Qiagen).

Discard flow-through and place QIAquick column back in the same collection tube.

Add 0.5 mL of Buffer QG to QIAquick column and centrifuge for 1 min.

Add 0.75 mL of Buffer PE to QIAquick column and centrifuge for 1 min.

Discard the flow-through and centrifuge the QIAquick column for an additional 1 min at 17,900 rpm.

Place QIAquick column into a clean 1.5 mL microcentrifuge tube.

To elute DNA, add 35 μL of Buffer EB to the center of the QIAquick membrane and centrifuge the column for 1 min at 17,900 rpm (Buffer EB is 10 mM Tris-cl, pH 8.5).

Measure the DNA fragments concentration using the NanoDrop 2000 Spectrophotometer (Using EB buffer for the Blank measurement)

Step 3

Set up the ligation reaction in a 1.7 mL Eppendorf microcentrifuge tube on ice.

Use the pre-constructed spreadsheet (Concatemeric Ligations.xlsx) to calculate the volumes of each fragment that needs to be mixed to create about a 25:1 molar ratio of vector to PGK-ble.

Maximize the volume of fragments in the ligation reaction and maintain the desired molar ratio.

The T4 DNA Ligase Buffer should be thawed and re-suspended at room temperature (T4 DNA Ligase Buffer comprises the following components: 50 mM Tris-HCl, 10 mM MgCl2, 1 mM ATP, 10 mM DTT, pH 7.5).

Pipette the ligation reaction. In the above example, 90 μL of DNA mixture was utilized by adding 10 μL of 10×ligation buffer (NEB Quick Ligation kit), and 0.5 μL of Ligase enzyme (available from New England BioLabs).

Prepare the following reaction mixture containing at room temperature:

| Component | Vector |
|---|---|
| The vector fragment | |
| The ble-resistant fragment | |
| 10 × T4 DNA Ligase Buffer | 10 |
| T4 DNA Ligase | 0.5 |
| Water, nuclease-free | To 90 |
| Total Volume | 90 |

Mix gently by pipetting up and down.

Incubate at room temperature for overnight

Step 4

The concatemeric array was harvested and purified prior to transfection into GPRG cells by the silica-based membrane (DNeasy Blood & Tissue Kit).

Pipet the concatemeric array mixture into the DNeasy Mini spin column placed in a 2 mL collection tube.

Centrifuge at 8000×g for 1 min. Discard flow-through and collection tube.

Place the DNeasy Mini spin column in a new 2 mL collection tube (provided) (available from Qiagen).

Add 500 μL Buffer AW1, and centrifuge for 1 min at 8000×g.

Discard flow-through and collection tube

Place the DNeasy Mini spin column in a new 2 mL collection tube (provided)

Add 500 μL Buffer AW2, and centrifuge for 3 min at 20,000×g to dry the DNeasy membrane.

Discard flow-through and collection tube.

Place the DNeasy Mini spin column in a clean 1.7 mL Eppendorf microcentrifuge tube

Add 200 μL Buffer AE directly onto the DNeasy membrane.

Incubate at room temperature for 4 min

Centrifuge for 1 min at 8000×g to elute the DNA mixtures.

Repeat elution once

Measure the concatemeric DNA concentration by the NanoDrop Lite Spectrophotometer

### Example 4—Protocol for Generating Producer Cell Lines Using a Concatemeric Array

Passage the cells at least 4 times after thawing before using them in the viral vector production.

Ensure that cells are healthy and greater than 95% viable before vectors induction using Trypan Blue method (Trypan Blue is commonly used in dye exclusion procedures for viable cell counting. This method is based on the principle that live (viable) cells do not take up certain dyes, whereas dead (non-viable) cells do. Staining facilitates the visualization of cell morphology).

Culture the desired quantity of GPRG cells.

Subculture the cells at least two times daily passage before seed the cells.

The first day, remove the medium of the GPRG cell lines culture dish and wash the cells with 1×PBS.

Gently Pipette 1×TrypLE Express onto the washed cell monolayer using 3 ml for T75 flask or 1 mL for T25 Flask.

Rotate flask to cover the monolayer with TrypLE Express.

Return flask to the incubator and leave for 2 minutes.

Gently tapped side of the flasks to release any remaining attached cells.

Re-suspend the cells in 2 mL of the fresh D10 medium and transfer to a 15 mL conical centrifuge tube.

Centrifuge cells for 5 minutes at 1200 rpm.

Aspirate medium and gently suspend pellet in 5 mL fresh D10 culture medium with Doxycycline (1 ng/mL)

Determine the cell counts by TC10™ Automated Cell Counter

Seed the cells 20-24 hours before the transfection at 80% confluent at culture dish (by plating 3.2×10⁶ Live Cells in 60-mm culture dish with Doxycycline)

Prepare the concatemeric arrays formation (see, for example, Example 3).

On the second day, allow CalPhos™ Mammalian Transfection Kit to come to room temperature prior to the transfection (available From ClonTech).

Purify the concatemeric DNA and measure the concentration (the concatemeric array may be purified according to the methods described herein)

Prepare the transfection plasmid DNA (4 mL, 60 mm culture dish).

For each transfection, prepare Solution A and Solution B in the separate 15 mL conical centrifuge tube

Bubbling Solution B (2×HBS) by Pipette-aids and add the Solution A (DNA mixture) drop by drop.

Incubate the transfection solution at room temperature for 15 min

Gently add the transfection solution to the culture dish.

Gently move plates back and forth to distribute transfection solution evenly.

Incubate plates at 37° C. for 4 hours in a CO₂ incubator.

Warm-up 5 mL fresh D10 media per 60 mm culture dish in a 37° C. CO₂ incubator

After 4 hours wash with 1 mL pre-warmed D10 and change with 4 mL pre-warmed fresh D10 medium

Incubate at the 5% CO₂ 37° C. incubator

48 hours after concatemer transfection, harvest the transfected GPRG cells (Perform a sub-culturing of cells).

Re-plate the cells in the T150 flask or the 30 mL, 150 mm culture dish with fresh D10 media containing Zeocin (50 μg/mL) and Doxycycline (1 ng/mL)

Feed the cells with selective medium (Zeocin, 50 μg/mL) with Doxycycline (1 ng/mL) every 3-4 days until the cell foci are identified (usually observed within 1-2 weeks)

### Example 5—Description of Cell Lines and Sequences Used to Generate the GPRG Packaging Cell Line

HEK-293T/17 are a sub-clone of HEK-293T. These cells stably express SV-40 T antigen, and a particular clone was selected specifically for its high transfectability. A master cell bank based on HEK-293T/17 was generated (HEK-293T/17 MCB).

SFG-IC-HIVgp-Ppac2 is a gamma retroviral vector that expresses codon-optimized HIV gagpol under control of the CMV promoter, with puromycin resistance. The plasmid (pSFG-IC-HIVgp-Ppac2) that was used to make this vector was constructed using the following components:

(1) pSFG tcLuc ECT3 is a derivative of a retrovirus vector backbone plasmid (SFG), adapted for regulated gene expression using the tetracycline-regulated promoter system (Lindemann, D., Patriquin, E., Feng, S., & Mulligan, R. C. Versatile retrovirus vector systems for regulated gene expression in vitro and in vivo. Mol. Med. 3, 466-476 (1997));

(2) CMV enhancer/promoter driven codon optimized HIV NL4-3 gagpol gene;

(3) PGK promoter driven puromycin resistance gene derived from pMSCVpac (Hawley, R. G., Lieu, F. H., Fong, A. Z., & Hawley, T. S. Versatile retroviral vectors for potential use in gene therapy. Gene Ther. 1, 136-138 (1994)).

Infection of the HEK-293T/17 MCB with the SFG-IC-HIVgp-Ppac2 retroviral vector produced the GP cell line.

SFG-tc-revco is a gamma retroviral vector that expresses codon-optimized HIV rev under control of the tetracycline responsive promoter. The plasmid used to produce this vector (pSFG-tc-revco) was constructed using the following components:

(1) The HIV rev gene based on the NL4-3 strain sequence as above, and

(2) pSFG tcLuc ECT3 (described above)

SFG-tTA is a gamma retroviral vector that expresses the chimeric transcriptional transactivator under control of the retroviral LTR (Lindemann, D., Patriquin, E., Feng, S., and Mulligan, R. C. Versatile retrovirus vector systems for regulated gene expression in vitro and in vivo. Mol. Med. 3, 466-476 (1997)). It is based on the SFG retroviral vector, an incorporates a Tet promoter element from plasmid pUHD15-1 (Gossen M, and Bujard, H. (1992) PNAS 89 12:5547-5551).

Infection of the GP cell line with SFG-tc-revco and SFG-tTA produced the GPR cell line

SFG-tc-VSVG is a gamma retroviral vector that expresses VSV glycoprotein G under control of the tetracycline-regulated promoter. The plasmid used to make this vector (pSFG-tc-VSVG) was generated using the same pSFGtcLucECT3 backbone as the other vectors, and plasmid pMD.G as a source of the VSVG envelope protein (see Ory, D. S., Neugeboren, B. A., and Mulligan, R. C. A stable human-derived packaging cell line for production of high titer retrovirus/vesicular stomatitis virus G pseudotypes. Proc. Natl. Acad. Sci. U.S.A 93, 11400-11406 (1996) and Rose, J. K. & Gallione, C. (1981) J. Virol. 39, 519-528).

Infection of the GPR cell line with SFG-tc-VSVG produced the GPRG cell line.

Infection of GPR cell line with Retro-SVGmu to generate GPRS cell line is described by Lee, Chi-Lin et al. “Construction of Stable Producer Cells to Make High-Titer Lentiviral Vectors for Dendritic Cell-Based Vaccination.” Biotechnology and Bioengineering 109.6 (2012): 1551-1560. PMC. Web. 14 Apr. 2016.

### Example 6—Concatemeric Array Purification

The concatemer was harvested and purified prior to transfection into GPRG cells by the silica-based membrane (DNeasy Blood & Tissue Kit).

Pipet the concatemeric array mixture into the DNeasy Mini spin column placed in a 2 mL collection tube.

Centrifuge at 6000×g for 1 min. Discard flow-through and collection tube

Place the DNeasy Mini spin column in a new 2 mL collection tube (provided).

Add 500 μL Buffer AW1, and centrifuge for 1 min at 6000×g.

Discard flow-through and collection tube.

Place the DNeasy Mini spin column in a new 2 mL collection tube (provided)

Add 500 μL Buffer AW2, and centrifuge for 3 min at 20,000×g to dry the DNeasy membrane.

Discard flow-through and collection tube.

Place the DNeasy Mini spin column in a clean 1.7 mL Eppendorf microcentrifuge tube.

Add 200 μL Buffer AE directly onto the DNeasy membrane.

Incubate at room temperature for 4 min.

Centrifuge for 1 min at 6000×g to elute the DNA mixtures.

Repeat elution once (add new elution buffer).

Measure the concatemeric DNA concentration by the NanoDrop Lite Spectrophotometer.

### Example 7—TL20-UbcGFP & Cal1-WPRE Producer Cell Line

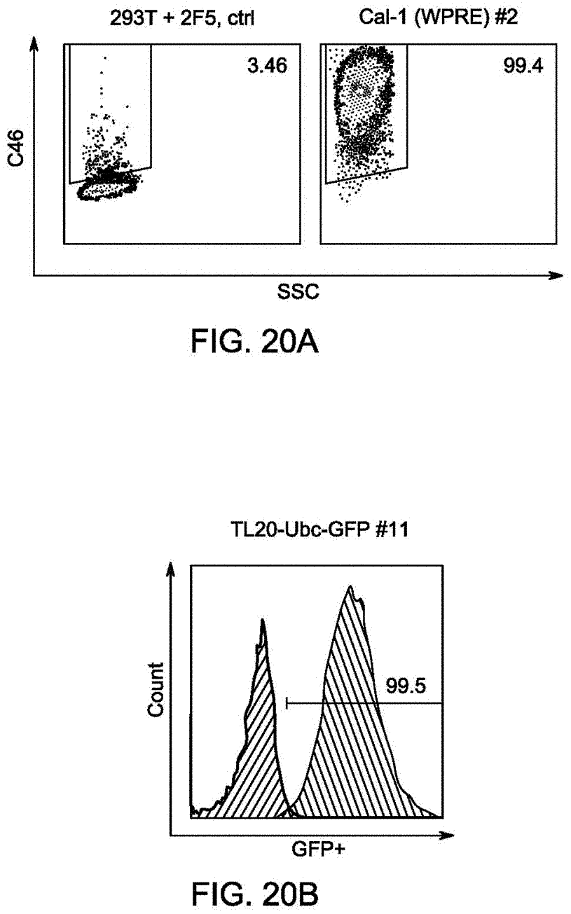

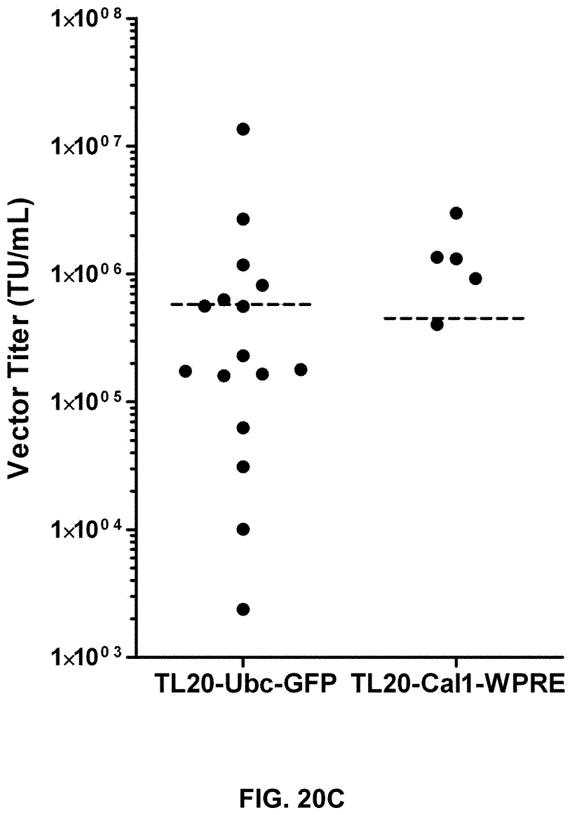

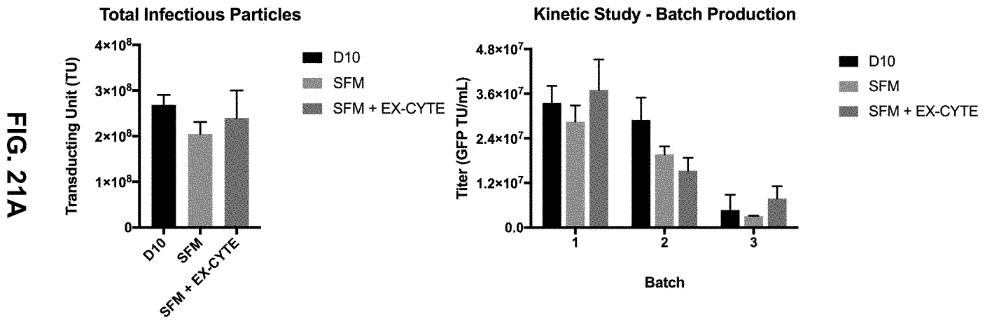

Table 4 which follows summarizes two stable producer cell line cells that were synthesized according to the methods describes herein. Data relating to the TL20-Cal1-wpre and TL20-Unc-GFP vectors is illustrated further in FIGS. 20A, 20B, and 20C.

TABLE 4
Comparison of two stable producer cell line cells prepared according to
the methods described herein

| Selection and Screening | TL20c-Ubc-GFP | TL20c-Cal1-WPRE |
|---|---|---|
| Method | Single Cell Sorting² | Single Cell Sorting² |
| Seed Cell Density | 1 cell/well | 1 cell/well |
| Culture Medium | Conditioned Medium | Conditioned Medium |
| Efficiency of clone | 28/96 | 22/96 |
| formation | | |
| Complete Expanded | 16 | 17 |
| Evaluated clones | 16 | 5 |
| Polyclonal Vector | 5.77 × 10⁵ TU/mL | 4.50 × 10⁵ TU/mL |
| Production | | |
| The productivity of the | 1.36 × 10⁷ TU/mL | 3.0 × 10⁶ TU/mL |
| best clones | | |

Abbreviation: TU, Transduction Unit
Performing single cell sorting by using flow cytometer at USC Flow Cytometry Core Facility
Conditioned Media: DMEM with GlutaMax;
FBS (10% w/v);
Pen/Strep (1% w/v);
Doxycycline (1 ng/mL)

Other Therapeutic Genes

In some embodiments, the synthesized vector may include any of the therapeutic genes enumerated below. For instance, nucleic acids encoding any of the genes enumerated below may be inserted in a recombinant plasmid as described herein. In some embodiments, the therapeutic gene corrects a single-gene disorder. In some embodiments, the therapeutic gene is used to treat immune deficiencies, hereditary diseases, blood diseases (e.g. hemophilia, hemoglobin disorders), lysosomal storage diseases, neurological diseases, angiogenic disorders, or cancer.

In some embodiments, the therapeutic gene is a gene encoding an enzyme adenosine deaminase, a gene encoding alpha-1-antitrypsin, a gene encoding a cystic fibrosis transmembrane conductance regulator, a gene encoding the enzyme Galactose-1-phosphate uridylyltransferase, a gene encoding a clotting factor (e.g. human Factor IX), a gene encoding a lipoprotein lipase gene, one or more genes encoding the enzymes required for dopamine synthesis, a gene encoding for glial cell line-derived neurotrophic factor (GDNF), a gene encoding interleukin-2 receptor subunit gamma (IL-2RG), a gene encoding Gp91phox, a gene encoding the Wiskott-Aldrich syndrome protein, a gene encoding a globin protein, a gene encoding a mutated globin protein (e.g. one having antisickling properties, a gene encoding a mutated beta-globin, a gene encoding gamma-globin, a gene encoding an anti-CD19 antibody, etc. In other embodiments, the therapeutic gene is selected from the group consisting of a globin gene, sphingomyelinase gene, alpha-L-iduronudase gene, huntingtin gene, neurofibromin 1 gene, MLH1 gene, MSH2 gene, MSH6 gene, PMS2 gene, cystic fibrosis transmembrane conductance regulator gene, hexosaminidase A gene dystrophin gene, FMR1 gene, phenylalanine hydroxylase gene and low-density lipoprotein gene.

Examples of classes of therapeutic genes include, but are not limited to, tumor suppressor genes, genes that induce or prevent apoptosis, genes encoding enzymes, genes encoding antibodies, genes encoding hormones, genes encoding receptors, and genes encoding cytokines, chemokines, or angiogenic factors. Specific examples of therapeutic genes include, but are not limited to, Rb, CFTR, p16, p21, p2′7, p5′7, p′73, C-CAM, APC, CTS-I, zacl, scFV, ras, DCC, NF-I, NF-2, WT-I, MEN-I, MEN-II, BRCA1, VHL, MMAC1, FCC, MCC, BRCA2, IL-I, IL-2, IL-3, IL-4, IL-5, IL-6, IL-7, IL-8, IL-9, IL-I0, IL-11 IL-12, IL-15Ra, IL-15, IL-21, GM-CSF, G-CSF, thymidine kinase, mda7, FUS1, interferon alpha, interferon beta, interferon gamma, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TALI, TCL3, YES, MADH4, RB1, TP53, WT1, TNF, BDNF, CNTF, NGF, IGF, GMF, aFGF, bFGF, NT3, NTS, ApoAI, ApoATV, ApoE, RaplA, cytosine deaminase, Fab, ScFv, BRCA2, zacl, ATM, HIC-I, DPC-4, FHIT, PTEN, ING1, NOEY1, NOEY2, OVCA1, MADR2, 53BP2, IRF-I, zacl, DBCCR-I, rks-3, COX-I, TFPI, PGS, Dp, E2F, ras, myc, neu, raf, erb, fins, trk, ret, gsp, hst, abl, ElA, p300, VEGF, FGF, thrombospondin, BAI-I, GDAIF, MCC, 41BBL, CD80, CD86, or OX40.

Other examples of therapeutic genes are the tumor suppressor genes including, but not limited to, FUS1, Gene 26 (CACNA2D2), PL6, LUCA-I (HYAL1), LUCA-2 (HYAL2), 123F2 (RASSF1), 101F6, Gene 21 (NPRL2), SEM A3, NF1, NF2, and p53.

Yet other examples of therapeutic genes are genes encoding enzymes including, but not limited to, ACP desaturase, ACP hydroxylase, ADP-glucose pyrophorylase, PDE8A (camp Phosphodiesterase), ATPase, alcohol dehydrogenase, amylase, amyloglucosidase, catalase, cellulase, cyclooxygenase, decarboxylase, dextrinase, esterase, DNA polymerase, RNA polymerase, hyaluron synthase, galactosidase, glucanase, glucose oxidase, GTPase, helicase, hemicellulase, hyaluronidase, integrase, invertase, isomerase, kinase, lactase, lipase, lipoxygenase, lyase, lysozyme, pectinesterase, a peroxidase, a phosphatase, a phospholipase, a phosphorylase, polygalacturonase, proteinase, peptidase, pullanase, recombinase, reverse transcriptase, topoisomerase or xylanase. Further examples of therapeutic genes include the genes encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerbrosidase, sphingomyelinase, alpha-L-idurom dase, glucose-6-phosphate dehydrogenase, HSV thymidine kinase, or human thymidine kinase.

Further examples of therapeutic genes include genes encoding hormones including, but not limited to, growth hormone, prolactin, placental lactogen, luteinizing hormone, follicle-stimulating hormone, chorionic gonadotropin, uiyroid-stimulating hormone, leptin, adrenocorticotropin, angiotensin I, angiotensin II, alpha-endorphin, beta-melanocyte stimulating hormone, cholecystokinin, endothelin I, galanin, gastric inhibitory peptide, glucagon, insulin, lipotropins, neurophysins, somatostatin, calcitonin, calcitonin gene related peptide, beta-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

Yet other therapeutic genes may be incorporated into an expression, including those genes described below.

Adenosine Deaminase-Severe Combined Immunodeficiency (ADA-SCID) deficiency results in the accumulation of toxic metabolites that destroy the immune system, causing severe combined immunodeficiency (ADA-SCID), often referred to as the “bubble boy” disease. In some embodiments, the second nucleic acid of the expression vectors described herein encodes for the human ADA cDNA sequence.

Severe Combined Immunodeficiency (SCID-X1) Disease is the most common form of SCID, accounting for 40-50% of SCID cases reported worldwide. Mutations in the IL2RG gene are leads to defective expression of the common gamma chain (γc), a subunit shared by a host of cytokine receptors, including interleukin (IL)-2, 4, 7, 9, 15, and 21 receptor complexes, which play a vital role in lymphocyte development and function. In some embodiments, the second nucleic acid of the expression vectors described herein encodes the human yc cDNA sequence.

Chronic granulomatous disease (CGD) is caused by defects in the subunits (gp91phox, p22phox, p47phox, p40phox or p67phox) of the phagocyte-derived NADPH oxidase. Mutations in the CYBB gene—encoding the gp91phox subunit—are responsible for the X-linked form of CGD, which accounts for approximately 70% of patients. X-linked CGD is characterized by severe, life-threatening bacterial and fungal infections due to an impaired production of superoxide anions and other reactive oxygen intermediates by neutrophils, eosinophils, monocytes and macrophages. Another aspect of the disease is the sterile, chronic, granulomatous inflammation affecting organs such as the gut or lung, mainly caused by increased production of pro-inflammatory cytokines, delayed apoptosis of inflammatory cells and deficient secretion of anti-inflammatory mediators by activated neutrophils. The poor outcome is associated with a history of invasive fungal infection, liver abscesses and chronic granulomatous inflammation. Available therapeutic strategies include antibiotic long-life prophylaxis, IFN-γ administration, and HCT. In some embodiments, the second nucleic acid of the expression vectors described herein encodes the human subunit cDNA sequence.

Metachromatic leukodystrophy (MLD) MLD is a rare autosomal-recessive lysosomal storage disease caused by mutations in the arylsulfatase A (ARSA) gene that result in enzyme deficiency and accumulation of the undegraded substrate cerebroside 3-sulphate (sulphatide) in neural and glial cells in the central nervous system and peripheral nervous system. This accumulation of sulphatide leads to progressive demyelination and neurodegeneration. In some embodiments, the second nucleic acid of the expression vectors described herein encodes the human ARSA cDNA sequence.

Mucopolysaccharidosis I (MPS-I) or Hurler syndrome is a lysosomal storage disorder caused by a deficiency of the alpha-L-iduronidase enzyme (IDUA). The disease is characterized by inappropriate storage of glycosamminoglycans (GAGs) with accompanying organ enlargement and damage, excretion of abnormal quantities of GAGs in urine, and disrupted GAG turnover that especially affects connective tissues. Clinical manifestations include skeletal abnormalities, hepatosplenomegaly, mental retardation, and cardiovascular and respiratory dysfunction. IDUA deficiency can result in a wide range of phenotypic presentations, and MPS I Hurler (MPS IH) represents the most severe disease variant within this spectrum, characterized by a chronic, progressive, and disabling disease course involving multiple organs and the central nervous system. The disease is fatal in childhood if untreated, with death usually occurring within the first decade of life because of cardiorespiratory failure. In some embodiments, the second nucleic acid of the expression vectors described herein encodes the human cDNA of alpha-iduronidase (IDUA).

Gaucher's disease is the most common of the lysosomal storage diseases. It is an autosomal recessive lysosomal storage disease, caused by deficiency of the enzyme glucocerebrosidase (GBA), required for the degradation of glycosphingolipids. Clinical manifestations include hepatosplenomegaly, thrombocytopenia, bone disease and a bleeding diathesis, frequently resulting in presentation to hematologists. Gene therapy represents a therapeutic alternative for patients to enzyme replacement therapy and those lacking a suitable bone marrow donor. In some embodiments, the second nucleic acid of the expression vectors described herein encodes the human cDNA of the GBA gene.

Lysosomal storage diseases (LSDs) are rare inherited metabolic disorders characterized by a dysfunction in lysosomes. LSDs encompass approximately 70 genetically distinct diseases, with a collective incidence of 1:5000 live births. Examples include Fabry disease (alpha-galactosidase A deficiency), Pompe disease (α-glucosidase [GAA] deficiency), Hunter syndrome (iduronate-2-sulfatase [I2S] deficiency), Sanfilippo syndrome (deficiency in one of the enzymes needed to break down the glycosaminoglycan heparan sulfate) and Krabbe disease (gal-actocerebrosidase deficiency). Likewise, inherited metabolic disorders are one cause of metabolic disorders, and occur when a defective gene causes an enzyme deficiency. It is believed that an expression vectors of the present disclosure may be adapted to incorporate a second nucleic acid sequence which encodes a gene suitable for use in treating any of the above-identified conditions.

Pyruvate kinase deficiency (PKD) is a monogenic metabolic disease caused by mutations in the PKLR gene that leads to hemolytic anemia of variable symptomatology and that can be fatal during the neonatal period. PKD recessive inheritance trait and its curative treatment by allogeneic bone marrow transplantation provide an ideal scenario for developing gene therapy approaches. In some embodiments, the second nucleic acid of the expression vectors described herein encodes the human PKLR cDNA.

Adrenoleukodystrophy (ALD) is a rare X-linked metabolic disorder caused by mutations in the ABCD1 gene which result in a deficiency in adrenoleukodystrophy protein (ALDP) and subsequent accumulation of very long chain fatty acids (VLCFA). VLCFA accumulation occurs in plasma and all tissue types but primarily affects the adrenal cortex and white matter of the brain and spinal cord, leading to a range of clinical outcomes. The most severe form of ALD, the inflammatory cerebral phenotype known as cerebral ALD (CALD), involves a progressive destruction of myelin, the protective sheath of the nerve cells in the brain that are responsible for thinking and muscle control. Symptoms of CALD usually occur in early childhood and progress rapidly if untreated, leading to severe loss of neurological function and eventual death in most patients. In some embodiments, the second nucleic acid of the expression vectors described herein encodes the human adrenoleukodystrophy protein (ALDP).

Fanconi anemia (FA) is an inherited bone marrow failure syndrome. A defect in 1 of at least 16 DNA repair genes leads to aplasia and enhanced risk for malignancies, especially AML and MDS. Additionally, the risk for adenoma, adenocarcinomas and squamous cell carcinomas is increased. Most patients also have a short stature, various morphological abnormalities and developmental disorders. Supportive treatment includes regular transfusions of blood products and growth hormone substitution due to concomitant endocrinopathies in FA patients. HSCT in the donor-matched setting has been the only curative option and is thus an attractive option for gene therapy. Despite the heterogeneity in genes affected, more than 60% of the patients have mutations in the FANCA gene. In some embodiments, the second nucleic acid of the expression vectors described herein encodes the human FANCA cDNA.

In some embodiments, the synthesized vector includes a nucleotide sequence encoding an C1 esterase inhibitor protein. C1 esterase inhibitor proteins are described in U.S. Pat. No. 10,214,731 and in U.S. Patent Publication No. 2018/0334493, the disclosures of which are hereby incorporated by reference herein in their entireties.

In some embodiments, the synthesized vector includes a nucleotide sequence encoding Bruton's tyrosine kinase (BTK) for treating X linked agammaglobulinemia (XLA). BTK is an enzyme that in humans is encoded by the BTK gene. BTK is a kinase that plays a crucial role in B-cell development. For example, BTK plays a crucial role in B cell maturation as well as mast cell activation through the high-affinity IgE receptor. Mutations in the BTK gene are implicated in the primary immunodeficiency disease X-linked agammaglobulinemia (Bruton's agammaglobulinemia). Patients with XLA have normal pre-B cell populations in their bone marrow but these cells fail to mature and enter the circulation. In some embodiments, the synthesized vector includes a nucleotide sequence which restores BTK expression. Suitable vectors are described in PCT Publication No. WO/2018/195297, the disclosure of which is incorporated by reference herein in its entirety.

In some embodiments, the synthesized vector includes one or more nucleotide sequences encoding gene or components for correcting a primary immunodeficiency (see Farinelli G., et al. (2014) Lentiviral vectors for the treatment of primary immunodeficiencies. J Inherit Metab Dis. 37:525-33, the disclosure of which is hereby incorporated by reference herein in its entirety).

In some embodiments, the synthesized vector includes a nucleotide sequence encoding a nuclease. In some embodiments, the synthesized vector may include a nucleotide sequence encoding a homing endonuclease (e.g. 1-Sce1, 1-Ceu1, F1-Psp1, F1-Sce, 1-SceTV, I-Csm1, 1-Pan1, 1-Scell, 1-Ppol, 1-Scell1, 1-Cre1, 1-Tev1, and 1-Tev11), a transcription activator-like effector nuclease (TALEN), a zinc finger nuclease (ZFN), a Type II clustered regularly interspaced short palindromic repeats (CRISPR) associated (Cas) nuclease, or a megaTAL nuclease, including any of those described in PCT Publication Nos. WO/2018/034523, WO/2017/156484, WO/2017/106528, and WO/2015/089046 or in US Patent Publication No. US/2019/0169597, the disclosures of which are each hereby incorporated by reference herein in their entireties.

In some embodiments, the synthesized vector may include a nucleotide sequence encoding an enzyme that may exhibit at least endonuclease activity. In some embodiments, the synthesized vector may include a nucleotide sequence encoding CRISPR/Cas components, e.g. Cas proteins or CRISPR-associated proteins. In some embodiments, the Cas proteins include Cas9 proteins, Cas9-like proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, Cas12 proteins (e.g. such as Cas12a, Cas12b, Cas12c, Cas12d, Cas12e), and variants and modifications thereof. In some embodiments, the Cas 9 proteins include Cas9 polypeptides from any of a variety of biological sources, including, e.g., prokaryotic sources such as bacteria and archaea. Bacterial Cas9 includes, Actinobacteria (e.g., Actinomyces naeslundii) Cas9, Aquificae Cas9, Bacteroidetes Cas9, Chlamydiae Cas9, Chloroflexi Cas9, Cyanobacteria Cas9, Elusimicrobia Cas9, Fibrobacteres Cas9, Firmicutes Cas9 (e.g., Streptococcus pyogenes Cas9, Streptococcus thermophilus Cas9, Listeria innocua Cas9, Streptococcus agalactiae Cas9, Streptococcus mutans Cas9, and Enterococcus faecium Cas9), Fusobacteria Cas9, Proteobacteria (e.g., Neisseria meningitides, Campylobacter jejuni and lari) Cas9, Spirochaetes (e.g., Treponema denticola) Cas9, and the like. Archaea Cas 9 includes Euryarchaeota Cas9 (e.g., Methanococcus maripaludis Cas9) and the like.

In some embodiments, the synthesized vector includes a nucleotide sequence encoding a mammalian β globin gene (HBB), a gamma globin gene (HBG1), a B-cell lymphoma/leukemia 1 1A (BCL1 1A) gene, a Kruppel-like factor 1 (KLF1) gene, a CXCR4 gene, a PPP1R12C (AAVS 1) gene, an albumin gene, and a Leucine-rich repeat kinase 2 (LRRK2) gene.

All publications mentioned in this specification are herein incorporated by reference in their entirety. It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A method of harvesting vector supernatant from stable producer cell line cells comprising: inducing viral vector production from the stable producer cell line cells; and repeatedly harvesting the viral vectors from the induced stable producer cell line cells in serum-free media every about 40 to about 56 hours following an initial harvesting of the viral vectors.

2. The method of claim 1, wherein the method provides for a production of viral titer ranging from about 0.5×10⁶ TU/mL to about 4×10⁶ TU/mL during each individual harvesting of the repeated harvesting.

3. The method of claim 2, wherein the viral titer ranges from about 0.5×10⁶ TU/mL to about 2×10⁶ TU/mL during each individual harvesting of the repeated harvesting.

4. The method of claim 2, wherein the viral titer ranges from about 0.5×10⁶ TU/mL to about 1.5×10⁶ TU/mL during each individual harvesting of the repeated harvesting.

5. The method of claim 1, wherein the viral vectors are harvested at least 5 times.

6. The method of claim 1, wherein the viral vectors are harvested at least 10 times.

7. The method of claim 1, wherein the viral vectors are harvested at least 20 times.

8. The method of claim 1, wherein the repeated harvesting occurs for a period of time ranging from between about 10 days to about 90 days.

9. The method of claim 1, wherein the repeated harvesting occurs for a period of time ranging from between about 20 days to about 70 days.

10. The method of claim 1, wherein the repeated harvesting comprises adding fresh serum-free media to the induced generated stable producer cell line cells without introducing additional generated stable producer cell line cells.

11. The method of claim 1, wherein the stable producer cell line cells are passaged in serum-containing media; and wherein the cells are cultured in serum-free media.

12. The method of claim 1, wherein the stable producer cell line cells are passaged in serum-containing media; and wherein the cells are cultured in serum-containing media.

13. The method of claim 1, wherein the initial harvesting of the viral vectors occurs at least about 40 hours after induction.

14. The method of claim 1, wherein the viral vectors are repeatedly harvested about every 48 hours.

15. The method of claim 1, wherein the serum-free media comprises one or more additives.

16. The method of claim 1, wherein the viral vectors comprise a nucleic acid sequence encoding a therapeutic gene.

17. The method of claim 16, wherein the therapeutic gene corrects for sickle cell disease or at least mitigates one symptom of a sickle cell disease.

18. The method of claim 16, wherein the therapeutic gene is selected from the group consisting of a gamma-globin gene, a C1 esterase inhibitor protein, Bruton's tyrosine kinase, and a Wiskott-Aldrich Syndrome protein.

19. The method of claim 1, wherein the viral vectors comprise a nucleic acid sequence encoding an RNAi to knockdown HPRT or CCR5.

20. The method of claim 1, wherein the viral vectors comprise (i) a first nucleic acid sequence encoding an RNAi to knockdown HPRT, and (ii) a second nucleic sequence encoding a therapeutic gene.

21. The method of claim 1, wherein the stable producer cell line cells are derived from one of a GPR, GPRG, GPRT, GPRGT, or GPRT-G packaging cell line or a derivative thereof.

22. The method of claim 1, wherein the stable producer cell line cells are generated by (a) synthesizing a vector by cloning one or more genes into a recombinant plasmid; (b) forming a concatemeric array from (i) an expression cassette excised from the synthesized vector, and (ii) an expression cassette obtained from an antibiotic resistance cassette plasmid; (c) transfecting packaging cell line cells selected from the group consisting of GPR, GPRG, GPRT, GPRGT, and GPRT-G with the formed concatemeric array; and (d) isolating the stable producer cell line cells.

23. The method of claim 22, wherein the recombinant plasmid comprises a nucleotide sequence having at least about 90% identity to that of SEQ ID NO: 1.

24. The method of claim 22, wherein the recombinant plasmid comprises a nucleotide sequence having at least about 80% identity to that of SEQ ID NO: 2.

25. The method of claim 22, wherein the recombinant plasmid comprises a nucleotide sequence having at least about 90% identity to that of SEQ ID NO: 2.

26. The method of claim 22, wherein the recombinant plasmid comprises a nucleotide sequence encoding a packaging signal; a nucleotide sequence encoding a central polypurine tract; a nucleotide sequence encoding a Rev response element; and a nucleotide sequence encoding a self-inactivating long terminal repeat.

27. The method of claim 22, wherein the synthesized vector comprises a nucleic acid sequence encoding a shRNA to knockdown hypoxanthine phosphoribosyltransferase (“HPRT”).

28. The method of claim 22, wherein the synthesized vector comprises a nucleic acid sequence encoding a therapeutic gene.

29. The method of claim 28, wherein the therapeutic gene is selected from the group consisting of a gamma-globin gene, a C1 esterase inhibitor protein, Bruton's tyrosine kinase, and a Wiskott-Aldrich Syndrome protein.


SEQUENCE LISTING (15 sequences)

SEQ ID NO: 1 - DNA, 6565 bp, Artificial Sequence
  Synthetic PUC57-TL20C
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 2 - DNA, 3901 bp, Artificial Sequence
  Synthetic TL20C vector backbone
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagct

SEQ ID NO: 3 - DNA, 343 bp, Artificial Sequence
  Synthetic nucleotide sequence encoding the packing signal
agtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcag

SEQ ID NO: 4 - DNA, 477 bp, Artificial Sequence
  Synthetic Central polypurine tract (cppt)
aattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccata

SEQ ID NO: 5 - DNA, 769 bp, Artificial Sequence
  Synthetic nucleotide sequence encoding the rev response element
aggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccg

SEQ ID NO: 6 - DNA, 181 bp, Artificial Sequence
  Synthetic nucleotide sequence encoding the self-inactivating long terminal repeat
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 7 - DNA, 25 bp, Artificial Sequence
  Synthetic nucleotide sequence encoding the multiple cloning site
ttcgaacgcgtgcggccgcatcgat

SEQ ID NO: 8 - DNA, 1978 bp, Artificial Sequence
  Synthetic LVSH5/C46
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacggatccccgagcaagctcagtttacaccttgtccgacggtgtaaactgagcttgctctttttgagacgagtcctcgagccataaagatggttaattaacccacccaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttggtaccgagctcggatccgccaccatgggagcaggagcaaccggaagggcaatggacggaccaagattgttacttctgctcctgctaggcgtgagcctgggaggagcaaggagctggatggagtgggacagggagatcaacaactacaccagcctgatccacagcctgatcgaggagagccagaaccagcaggagaagaacgagcaggagctgctggagctggacaagtgggccagcctgtggaactggttccggagcgagcggaagtgctgcgtggagtgcccaccatgcccagcaccaccagtggcaggacccctgatcgcactggtgaccagcggagccctgctggccgtgctgggcatcacaggctacttcctgatgaacaggaggagctggagcccaaccggagagcggctggagctggagccatga

SEQ ID NO: 9 - DNA, 1113 bp, Artificial Sequence
  Synthetic PGK-BLE SEQUENCE
ccagcctttggaattcctgcaggatgggattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcaggggcggggcgggcgcccgaaggtcctccggaggcccggcattctgcacgcttcaaaagcgcacgtctgccgcgctgttctcctcttcctcatctccgggcctttcgacctggatcctgcagcacgtgttgacaattaatcatcggcatagtatatcggcatagtataatacgactcactataggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatgctttatttgtgaaatttgtgatgctattgctttatttgtaaccattataagctgcaataaacaagttaacaacaacaattgcattcattttatgtttcaggttcagggggaggtgtgggaggttttttaaactagtgagtcgtattacccagcctttggg

SEQ ID NO: 10 - DNA, 288 bp, Artificial Sequence
  Synthetic (7tetO) doxycycline repressible promoter
tttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaa

SEQ ID NO: 11 - DNA, 98 bp, Artificial Sequence
  Synthetic HIV LTR R5 region
gggtctctctggttagaccagatctgagcctgggagctctctggctaactagggaacccactgcttaagcctcaataaagcttgccttgagtgcttca

SEQ ID NO: 12 - DNA, 83 bp, Artificial Sequence
  Synthetic HIV LTR U5 region
agtagtgtgtgcccgtctgttgtgtgactctggtaactagagatccctcagacccttttagtcagtgtggaaaatctctagca

SEQ ID NO: 13 - DNA, 412 bp, Artificial Sequence
  Synthetic chicken HS4 400 bp chromatin insulator
atccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcccc

SEQ ID NO: 14 - DNA, 449 bp, Artificial Sequence
  Synthetic rabbit beta-globin polyadenylation signal
gatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 15 - DNA, 2664 bp, Artificial Sequence
  Synthetic pUC57 Plasmid portion
gtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc